SEQUENCE LISTING (20 sequences)

SEQ ID NO: 1 - DNA, 21 bp, Homo sapiens
gagactcctccgtacagtaat

SEQ ID NO: 2 - DNA, 7218 bp, Homo sapiens
gccccgcatcgtgcccggccccgtcgcggagatcccggacgaccgtcgcgggttgatggtcgcattccagatgtaaacagcttcagaagcctgacggtcatatggtagaatcactgtggactgagacccacctttctagacctgaagcccaggaggaggaagaggaggctggttggtaccatgggcataatgctctgaatcctagtctctcacctagtatgtgagcagtccctgcagatggcccatttggagatcttgacaaagcctcttctgtttccaatggggtttttggcgcattctcacagacttagatgaaactgtgatggccaccgcagggggcaggtgctgacatcgtccccagccctgtggctgttcatccggacatcatttccaacctcaatatctaaatgccacagtgctcttggagcaagttgggctggggaccactgttgccttttaagaccataaaaccatgggaaacgcagaaagtcaacatgtagagcacgagttttatggagaaaagcatgccagcctggggcgcaagcacacttcccgctccctgcgcctctcgcacaagacgcggaggaccaggcacgcttcctcggggaaggtgatccacaggaactccgaagtgagcacccgatccagcagcacccccagcatcccccagtccctggctgaaaatggcctggagcccttctcccaagatggtaccctagaagacttcgggagccccatctgggtggaccgagtggacatgggcttgagacctgtgtcttacactgactcttctgtcactcccagcgtagacagcagcatcgtcctcacagcagcctctgtgcagagcatgccagacactgaggagagcaggctttacggggatgacgctacatatttggctgagggaggcaggaggcagcattcctatacatccaatgggcccactttcatggagacggcgagctttaagaagaaacgctccaaatctgcagacatctggcgggaggacagcctggaattctcactctctgatctgagccaagaacatttaacaagcaacgaagaaatcttgggttccgccgaagagaaggactgcgaggaggctcgggggatggaaacgcgggcgagtccgcggcagctcagcacctgtcagagagccaattccttgggtgacttgtatgctcagaaaaactctggagtgacagcaaacggggggccggggagcaaatttgcaggctactgtcggaatttggtgtctgatattcccaatcttgcaaaccataagatgccaccagctgctgctgaagagactcctccgtacagtaattataacacacttccctgtaggaaatctcactgtctctctgaaggtgccaccaacccacaaattagccatagcaacagcatgcaaggcagaagagctaaaacaactcaggatgttaatgcaggcgagggcagtgagtttgcagacagtgggattgaaggggccactaccgacacggacctcctgtccaggcgatctaatgccaccaactccagctactcacccaccacaggccgggcctttgtgggcagcgacagcggcagcagctccaccggggatgcggctcgtcagggggtgtacgagaacttccggcgggagctggagatgagcaccaccaacagcgagagcctggaggaggccggctcggcgcacagcgatgagcagagcagcggcaccctgagctctccgggccagtcggacatcctgctgaccgccgcacagggcacggtgcgcaaggccggcgccctggccgtcaagaacttcctggtgcacaagaagaacaagaaggtggagtcagccacccggaggaagtggaagcactactgggtgtccctgaaaggatgcacgctatttttctacgagagcgacggcaggtctgggatagaccacaacagcatccccaaacacgccgtctgggtggagaacagcattgtgcaggcggtgcctgagcaccccaagaaggactttgtcttctgcctcagcaattccctgggtgatgccttcctttttcagaccactagccagacggagcttgaaaactggatcaccgccatccactctgcctgcgccactgcggtcgcgaggcaccaccacaaggaagacacgctccgactcctgaaatcagagatcaaaaaactggaacagaagattgacatggatgaaaagatgaagaaaatgggtgaaatgcagctgtcttcagtcactgactcaaagaaaaagaaaacaatattagatcagatctttgtctgggagcaaaatctcgagcagttccaaatggacctgtttcgtttccgctgttatttagccagccttcagggtggggagctgccaaaccccaaaaggcttctcgcttttgcaagtcgaccaacgaaagtggccatgggccgccttggaatcttttcggtatcatcgtttcatgccctggtggcagcacgcactggtgaaactggagtgagaagacgtactcaggccatgtccagatccgcgagcaagcgaaggagcaggttttcttctctgtggggtctggatactacctccaaaaagaagcagggacggccaagcatcaatcaggtgtttggagagggaaccgaagctgtaaagaaatctttagagggaatatttgatgacattgttccagatggcaagagggagaaagaagtggtcttacctaacgttcaccagcacaaccctgactgcgacatttgggtccacgagtatttcactccatcctggttctgtctgcccaataatcagcctgccctgacggtcgtccggccaggcgacactgcacgggacaccctggagctgatttgcaagacacatcaactggatcattctgctcattacctgcgcctgaaatttctaatagaaaacaaaatgcagctctatgttccacagcccgaggaagacatctatgagctgctgtacaaagaaattgaaatctgtccaaaagtcactcagagcatccacattgagaagtcagatacagctgctgatacttacgggttttcactttcttctgtggaagaagatggtattcgaaggctgtacgtgaatagtgtgaaggaaaccggtttagcttccaagaaaggcctgaaagcaggagatgagattcttgagatcaataatcgtgctgctgacgccctgaactcttctatgctcaaagatttcctctcacagccctcgctgggcctcctggtgaggacctaccccgagctggaggaaggagtggagctgctggaaagcccgccccaccgagtggacggccctgccgaccttggcgagagccccctcgcctttctcaccagcaacccagggcacagcctttgcagcgagcagggcagcagtgctgagaccgctccagaggagaccgaggggccagacttggaatcctcagatgagactgatcacagcagcaagagtacagaacaggtggccgcattttgccgcagtttgcatgagatgaacccctctgaccagagcccatctcctcaggactccacggggcctcagctggcgaccatgagacaactctcggatgcagataagctgcgcaaggtgatctgcgagctcctggagacggagcgcacctacgtgaaggatttaaactgtcttatggagagatacctaaagcctcttcaaaaagaaacttttctcacccaggatgagcttgacgtgctttttggaaatttaacggaaatggtagagtttcaagtagaattccttaaaactctagaagatggagtgagactggtacctgatttggaaaagcttgagaaggttgatcaatttaagaaagtgctgttctctctggggggatcattcctgtattatgctgaccgcttcaagctctacagtgccttctgcgccagccacacaaaagttcccaaggtcctggtgaaagccaagacagacacggctttcaaggcattcttggatgcccagaacccgaagcagcagcactcatccacgctggagtcgtacctcatcaagcccatccagaggatcctcaagtacccacttctgctcagggagctgttcgccctgaccgatgcggagagcgaggagcactaccacctggacgtggccatcaagaccatgaacaaggttgccagtcacatcaatgagatgcagaaaatccatgaagagtttggggctgtgtttgaccagctgattgctgaacagactggtgagaaaaaagaggttgcagatctgagcatgggagacctgcttttgcacactaccgtgatctggctgaacccgccggcctcgctgggcaagtggaaaaaggaaccagagttggcagcattcgtcttcaaaactgctgtggtccttgtgtataaagatggttccaaacagaagaagaaacttgtaggatctcacaggctttccatttatgaggactgggaccccttcagatttcgacacatgatccccacggaagcgctgcaggttcgagctttggcgagtgcagatgcagaggcaaatgccgtgtgtgaaattgtccatgtaaaatccgagtctgaagggaggccggagagggtctttcacttgtgctgcagctccccagagagccgaaaggatttcctaaaggctgtgcattcaatcctgcgtgataagcacagaagacagctcctcaaaaccgagagccttccctcatcccagcaatatgtcccttttggaggcaaaagattgtgtgcactgaagggggccaggccggccatgagcagggcagtgtctgccccaagcaagtctcttgggaggaggaggcggcggctggctcgaaacaggtttaccattgattctgatgccgtctccgcaagcagcccggagaaagagtcccagcagccccccggtggtggggacactgaccgatgggtagaggagcagtttgatcttgctcagtatgaggagcaagatgacatcaaggagacagacatcctcagtgacgatgatgagttctgtgagtccgtgaagggtgcctcagtggacagagacctgcaggagcggcttcaggccacctccatcagtcagcgggaaagaggccggaaaaccctggatagtcacgcgtcccgcatggcacagctcaagaagcaagctgccctgtcggggatcaatggaggcctggagagcgcaagcgaggaagtcatttgggttaggcgtgaagactttgccccctccaggaaactgaacactgagatctgactgcgtcacctgccccgtagagaatgtgtgtagatacttcctgccctaactctgcccaccctcctgtaccgtcgacaagaatgtccccttaggtcgcgctcttgcacacacggttttggcagctgacttggttctgaagccatgtagccacccaactttgtcattttcaacaacatcagaaagaattgatcagaatcccaaataagcttgagtcctatcttctgtatattactaagggcttttatttattctcaataaatcagggcctgaacaattaaaagaaaaaagattctatagcactggaaagcaaatcaccccaggagttaacggatgtacaacagattaatttaagggatagtagcacacacacgatccttctatctgaaatcagtctcctagctggggaaacctctttcacacacaaaatgaaatgtgtacagcttgccgtgttctgactgtacccttccctcttccatgtctgagaatctccgtgtattttaagaatgtgtgaggagagggtggcgattcatgtttcaatgagcctctttttttttttccttcctgttttggtctatggctggtcttactctgtgtccatgttcggaagctctagttttgcatagaattatagagatgccaaactctttgaaaagagatccaaatttatcgcttgagagaaagaaaagaaacactattttttgtattttacctgagatacaggggcacaaatagatgagaattttacagtgttagtgtatgtatccctgagcctaaaaaatgaggatataaccttttacagagagagtgaggcgtggtggttttatatttatatatgaaaggccagcaagctcatgcgaaggatatacttttcttccaaaaagcggattttttttttttaatgtttgaatctatatttgagatgggagtttggttggattaaacatgacaccccggtgggcggtgtgtgtgtctgttgcacatggcagggaggggagcctccttctcatggggttgccatggtgatcattggtttttccatcaaaattgcatcttcatccatagattaccttccccttccctgacagtccataaccaaacctttaaacagaacaacctctttaaaaacttctcttgtgtttaacactttcttcatgccaacgaaacagggtaaacatgctcaaaacattaacagtctaaacagatatccaaatactaagaagaaaaacaagttatagcactttcaattttttttttttttttaaaaaaaggtttatagctttttcttttcccatgtcacaatgtccacttcctaagaagggtttaaaatactatgaaaactttctttttggggaaaatatctatttggtgtttgacacatcagtaggtactttaaagacctgaattttatagtagctttaggagttatattttataaaaatcagttatgactttatatttccagacaatagagagttcagtacatcatgctcttgtgcctctgcctgcttttcctgcgttcccaccctgtattccccccgcctttcgggtttccagggcttcgagcttgatcttttgaaagttttattctattaaatttttgctatatcttctggttttctgaaaaagctttagaatggtttctataccctttgtatcactgcatttttccatatcatctccggttcgatcgcgtccagatggaaaacggaagcagaggcttctaatcgtcgcatttactggctccagtgcaacacatccatctgaaaacactcggaagtctggtgcttggagagggtgccattgtctcttgtacataaggtcatgacgtgtctatgtcaaaagttcttatatatttcttttataagctgaaagaaggtctatttttatgtttttaggtctatgaatggaacgttgtaaatgcttgtcaaacaataaaaataacgaaaagtgaaaaaaaaaaaaaaaaaaaa

SEQ ID NO: 3 - protein, 1591 aa, Homo sapiens
MGNAESQHVEHEFYGEKHASLGRKHTSRSLRLSHKTRRTRHASSGKVIHRNSEVSTRSSSTPSIPQSLAENGLEPFSQDGTLEDFGSPIWVDRVDMGLRPVSYTDSSVTPSVDSSIVLTAASVQSMPDTEESRLYGDDATYLAEGGRRQHSYTSNGPTFMETASFKKKRSKSADIWREDSLEFSLSDLSQEHLTSNEEILGSAEEKDCEEARGMETRASPRQLSTCQRANSLGDLYAQKNSGVTANGGPGSKFAGYCRNLVSDIPNLANHKMPPAAAEETPPYSNYNTLPCRKSHCLSEGATNPQISHSNSMQGRRAKTTQDVNAGEGSEFADSGIEGATTDTDLLSRRSNATNSSYSPTTGRAFVGSDSGSSSTGDAARQGVYENFRRELEMSTTNSESLEEAGSAHSDEQSSGTLSSPGQSDILLTAAQGTVRKAGALAVKNFLVHKKNKKVESATRRKWKHYWVSLKGCTLFFYESDGRSGIDHNSIPKHAVWVENSIVQAVPEHPKKDFVFCLSNSLGDAFLFQTTSQTELENWITAIHSACATAVARHHHKEDTLRLLKSEIKKLEQKIDMDEKMKKMGEMQLSSVTDSKKKKTILDQIFVWEQNLEQFQMDLFRFRCYLASLQGGELPNPKRLLAFASRPTKVAMGRLGIFSVSSFHALVAARTGETGVRRRTQAMSRSASKRRSRFSSLWGLDTTSKKKQGRPSINQVFGEGTEAVKKSLEGIFDDIVPDGKREKEVVLPNVHQHNPDCDIWVHEYFTPSWFCLPNNQPALTVVRPGDTARDTLELICKTHQLDHSAHYLRLKFLIENKMQLYVPQPEEDIYELLYKEIEICPKVTQSIHIEKSDTAADTYGFSLSSVEEDGIRRLYVNSVKETGLASKKGLKAGDEILEINNRAADALNSSMLKDFLSQPSLGLLVRTYPELEEGVELLESPPHRVDGPADLGESPLAFLTSNPGHSLCSEQGSSAETAPEETEGPDLESSDETDHSSKSTEQVAAFCRSLHEMNPSDQSPSPQDSTGPQLATMRQLSDADKLRKVICELLETERTYVKDLNCLMERYLKPLQKETFLTQDELDVLFGNLTEMVEFQVEFLKTLEDGVRLVPDLEKLEKVDQFKKVLFSLGGSFLYYADRFKLYSAFCASHTKVPKVLVKAKTDTAFKAFLDAQNPKQQHSSTLESYLIKPIQRILKYPLLLRELFALTDAESEEHYHLDVAIKTMNKVASHINEMQKIHEEFGAVFDQLIAEQTGEKKEVADLSMGDLLLHTTVIWLNPPASLGKWKKEPELAAFVFKTAVVLVYKDGSKQKKKLVGSHRLSIYEDWDPFRFRHMIPTEALQVRALASADAEANAVCEIVHVKSESEGRPERVFHLCCSSPESRKDFLKAVHSILRDKHRRQLLKTESLPSSQQYVPFGGKRLCALKGARPAMSRAVSAPSKSLGRRRRRLARNRFTIDSDAVSASSPEKESQQPPGGGDTDRWVEEQFDLAQYEEQDDIKETDILSDDDEFCESVKGASVDRDLQERLQATSISQRERGRKTLDSHASRMAQLKKQAALSGINGGLESASEEVIWVRREDFAPSRKLNTEI

SEQ ID NO: 4 - DNA, 29 bp, Homo sapiens
cgggatccatgggaaacgcagaaagtcaa

SEQ ID NO: 5 - DNA, 18 bp, Homo sapiens
ccactttcgttgtcgact

SEQ ID NO: 6 - DNA, 18 bp, Homo sapiens
gagctgccaaaccccaaa

SEQ ID NO: 7 - DNA, 31 bp, Homo sapiens
atagtcgacgatctcagtgttcagtttcctc

SEQ ID NO: 8 - DNA, 36 bp, Homo sapiens
ataagaagcggccgcatgggaaacgcagaaagtcaa

SEQ ID NO: 9 - DNA, 55 bp, Homo sapiens
caccctttacaacaaatacccagatttcaagagaatctgggtatttgttgtaaag

SEQ ID NO: 10 - DNA, 55 bp, Homo sapiens
aaaactttacaacaaatacccagattctcttgaaatctgggtatttgttgtaaag

SEQ ID NO: 11 - DNA, 22 bp, Homo sapiens
aagacgtactcaggccatgtcc

SEQ ID NO: 12 - DNA, 20 bp, Homo sapiens
gacccaaatgtcgcagtcag

SEQ ID NO: 13 - DNA, 20 bp, Homo sapiens
gccataccagttaaacaggc

SEQ ID NO: 14 - DNA, 20 bp, Homo sapiens
gacctcagaagatgcactat

SEQ ID NO: 15 - DNA, 20 bp, Mus musculus
ctcccggtgaaagtgactga

SEQ ID NO: 16 - DNA, 20 bp, Mus musculus
gacctcagaagatgaactct

SEQ ID NO: 17 - DNA, 20 bp, Homo sapiens
ctgcaccaccaactgcttag

SEQ ID NO: 18 - DNA, 20 bp, Homo sapiens
ttcagctcagggatgacctt

SEQ ID NO: 19 - DNA, 25 bp, Mus musculus
tggaatcctgtggcatccatgaaac

SEQ ID NO: 20 - DNA, 25 bp, Mus musculus
taaaacgcagctcagtaacagtccg

(19) United States
(12) Patent Application Publication
(10) Pub. No.: US 2013/0137102 A1
(43) Pub. Date: May 30, 2013

(54) Methods and kits for modulating tumor invasiveness and metastatic potential

(71) Applicant: TUFTS MEDICAL CENTER, INC., Boston, MA (US)

(72) Inventor: Rachel J. Buchsbaum, Winchester, MA (US)

(73) Assignee: TUFTS MEDICAL CENTER, INC., Boston, MA (US)

(21) Appl. No.: 13/759,519

(22) Filed: Feb. 5, 2013

Related U.S. Application Data
(63) Continuation of application No. PCT/US2011/046546, filed on Aug. 4, 2011.
(60) Provisional application No. 61/371,165, filed on Aug. 5, 2010.

(51) Int. Cl. G01N 33/68; C12Q 1/02; C12Q 1/68

(52) U.S. Cl. CPC G01N 33/6872
USPC 435/6.11; 435/7.1; 435/6.12; 435/375; 435/455; 435/34; 506/9; 435/6.13

(57) ABSTRACT

Methods and kits for evaluating invasive potential and metastatic potential of cancers by assessing Tiam1 expression levels in fibroblasts in the microenvironments surrounding tumors are provided.

### RELATED APPLICATION

The present application claims the benefit of U.S. provisional application Ser. No. 61/371,165 filed Aug. 5, 2010, which is hereby incorporated by reference herein in its entirety.

### GOVERNMENT FUNDING

The invention was made with support from grant numbers NIH CA 095559 and CA12555 awarded by the National Institutes of Health. The United States government has certain rights in the technology.

### BACKGROUND OF THE TECHNOLOGY

The role of the microenvironment in the development of cancers such as breast cancer is gaining increased recognition [Cunha G R, et al., 1989, Cancer Treat Res 46:159-175]. There is growing evidence that the stromal microenvironment around cancer cells influences the growth, invasiveness, and metastatic behavior of cancer cells, and perhaps also therapeutic response. It is becoming increasingly apparent that there are bidirectional signals (termed co-evolution) between cancer cells and tumor-associated stroma. Tumor-associated stroma is comprised of various cell types, including fibroblasts, monocyte/macrophages, neutrophils, vascular cells, and bone marrow derived cells, along with extracellular molecules comprising or secreted into the extracellular matrix, such as collagens, fibronectin, VEGF and other growth factors, cytokines, and metalloproteinases. The list of factors that may participate in the co-evolution of tumors with tumor-associated stroma is growing, and the interplay between signaling pathways within the tumor cells and the stroma itself is just beginning to be understood [Li H, et al., 2007, J Cell Biochem 101:805-815; Bhowmick N A, et al., 2005, Curr Opin Genet Dev 15:97-101].

Since the discovery of oncogenes, proto-oncogenes, and their signaling pathways, significant effort has gone into the elucidation of the molecular pathways governing specific cellular behaviors and how these are corrupted in the tumor cells themselves. Much of this work has been done using traditional two-dimensional cell culture models, allowing for ready manipulation of individual signaling components. Some of the best-studied signaling molecules are the Ras family proteins, which function as molecular switches that control the flow of information from upstream inputs to downstream target pathways by cycling between active (GTP-bound) and inactive (GDP-bound) conformations [Boguski M S, et al., 1993, Nat 366:643-654]. The Rho sub-family proteins (Rho, Rac, and Cdc42) have been a particular focus of study since the identification of their roles in cytoskeleton dynamics [Cunha G R, et al., 1989, Cancer Treat Res 46:159-175] and are known to play key roles in multiple signaling pathways affected in malignant cell transformation. Three classes of regulatory proteins affect the activation state of Rho molecules: GEFs (guanine nucleotide exchange factors, which promote exchange of GTP for bound GDP and GTPase activation), GAPs (GTPase-activating proteins, which enhance intrinsic GTP-hydrolysis activity and GTPase inactivation), and GDIs (guanine-nucleotide dissociation inhibitors, which bind prenylated GDP-bound Rho proteins and allow translocation between membranes and cytosol). GEFs appear to be the primary regulators of Rho family activation in response to upstream stimuli [Erickson J W, et al., 2004, Biochemistry 43:837-842; Schmidt A, et al., 2002, Genes & Development 16:1587-1609; Rossman K L, et al., 2005, Nat Rev Mol Cell Biol 6:167-180].

There are more than 60 GEFs that have been identified for the Rho family proteins. The Rac GEF Tiam1 (T-cell lymphoma invasion and metastasis-inducing protein 1), first identified by retroviral mutagenesis as an invasion promoting factor in T-cell lymphomas, has since been recognized as a ubiquitous Rac activator with multiple effects in cells [Habets G G, et al., 1994, Cell 77:537-549, Mertens A E, et al., 2003, FEBS Lett 546:11-16]. Tiam1 and Rae have each been extensively studied for their functions within cancer cells themselves, and Tiam1 is increasingly being identified in human cancer cells. Increased Tiam1 expression is associated with increased invasiveness and/or epithelial-mesenchymal transition in colon, pancreatic, breast, and lung cancer cell lines [Minard M E, et al. 2005, Oncogene 24:2568-2573, Liu L, et al., 2005, World J Gastroenterol 11:705-707, Cruz-Monserrate Z, et al., 2008 Neoplasia 10:408-417, Minard M E, et al., 2004, Breast Cancer Res Treat 84:21-32, Hou M, et al., Acta Biochim Biophys Sin (Shanghai) 36:537-540]. Depletion of Tiam1 retards soft agar colony formation in a pancreatic cancer cell line, decreases growth and invasiveness of colorectal cancer cells, and decreases migration of oral cancer cells [Baines A T, et al., 2006, Methods Enzymol 407:556-574, Liu L, et al., 2006, Neoplasia 8:917-924, Supriatno, et al., 2003, Oncol Rep 10:527-532]. Tiam1 is a Wnt-responsive gene that is up-regulated in mouse intestinal tumors and human colon adenomas, yet germline knock-out leads to decreased growth of both skin and intestinal tumors in mouse models [Malliri A, et al., 2002, Nat 417:867-871, Malliri A, et al., 2006, JBC 281:543-548]. In human tumor specimens, higher levels of Tiam1 correlate with higher tumor grade, higher invasiveness, and/or poorer prognosis in human retinoblastoma, nasopharyngeal carcinoma, and prostate cancer [Minard M E, et al., 2004, Breast Cancer Res Treat 84:21-32, Adithi M, et al., 2006, Exp Eye Res 83:1446-1452, Cho W C, 2007, Mol Cancer 6:1, Engers R, et al., 2006, Br J Cancer 95:1081-1086]. However, Tiam1 expression is associated with a more favorable prognosis in human gastric cancer specimens, correlates inversely with invasiveness in renal carcinoma cell lines, and ectopic expression induces reversion of mesenchymal phenotype to epithelial phenotype in metastatic melanoma cells [Walch A, et al., 2008, Mod Pathol 21:544-552, Engers R, et al., 2001, JBC 276:41889-41897, Engers R, et al., 2000, Int J Cancer 88:369-376, Uhlenbrock K, et al., 2004, J Cell Sci 117(Pt 20):4863-4871]. Thus, Tiam1 induces paradoxical and opposing phenotypes in different malignancies and model systems. Notably, these studies have focused only on the role of Tiam1 within the malignant cells themselves.

For breast cancers and other cancers the major gains in disease control have come through improvements in screening and treatment of early stage disease. In many instances, once the cancer has invaded and metastasized, the treatments currently available are only palliative. As a result, the death rate of women with metastatic breast cancer has not significantly dropped. Thus, new strategies in identifying and treating and preventing potentially invasive and/or metastatic cancers such as breast cancer are needed. In cancers such as breast cancer, increased screening has identified women with early, non-invasive cancers. Only a portion of these cancers progress to invasive, and potentially metastatic, cancers. Currently available clinical diagnostic techniques can not differentiate which non-invasive cancers will become invasive. New methods in predicting invasive potential in early breast cancers are needed to more effectively tailor treatments.

### SUMMARY

An aspect of the invention provides a method for evaluating potential invasiveness of an epithelial cell cancer including: detecting Tiam1 in a tissue sample, such that the tissue sample includes fibroblasts and tumor cells or suspected tumor cells, and the detecting includes at least one of amount and location of the Tiam1; and, assessing the Tiam1 expression levels in fibroblasts adjacent to tumor cells, such that a decreased level of Tiam1 expression in the fibroblasts adjacent to tumor cells, in comparison to a control non-invasive standard or to a sample taken at a different point in time, is indicative of increased potential invasiveness of the epithelial cell cancer.

In an embodiment of this method, the method further including prior to detecting, obtaining the tissue sample from a subject having or suspected of having an epithelial cell cancer. In an embodiment of this method, the epithelial cell cancer is at least one selected from: breast, prostate, lung, bladder, uterine, ovarian, brain, head and neck, esophageal, pancreatic, gastric, germ cell, and colorectal cancers. In an embodiment of this method, the subject is at risk for developing an epithelial cell cancer. In an embodiment of this method, the subject is at risk for developing the cancer arising from family history or genetic analysis, or the patient is in remission from the cancer and is at risk for developing a recurrence of the cancer.

An embodiment of the invention provides a method for modulating invasiveness and metastatic potential of an epithelial cell cancer including contacting fibroblasts associated with the epithelial cell cancer with a reagent that causes increased expression of Tiam1. In an embodiment of this method, the reagent is selected from: a low molecular weight drug, a vector carrying a gene or a portion thereof encoding a Tiam1 protein, and a naked nucleic acid encoding the protein.

An embodiment of the invention provides a method for screening compounds to identify an agent that modulates invasiveness of an epithelial cell cancer including: contacting fibroblasts with at least one candidate compound, and assessing Tiam1 expression levels in resulting contacted fibroblasts, such that increased expression of Tiam1 in the contacted fibroblasts in comparison to fibroblasts not so contacted and otherwise identical, identifies the agent that modulates invasiveness of the epithelial cell cancer.

In an embodiment of the method, the fibroblasts are associated with tumor cells in situ in an animal, and the method further comprises a pre-clinical evaluation of the agent. In an embodiment of the method, the fibroblasts are associated with tumor cells in a three-dimensional spheroid system. In an embodiment of the method, the fibroblasts are associated with the epithelial cell cancer. In an embodiment of the method, the fibroblasts are in vitro.

In an embodiment of the method, the method further includes, prior to contacting, stressing the fibroblasts wherein the fibroblasts develop senescence. In an embodiment of the method, stressing comprises exposing the fibroblasts to an agent selected from: an oxidizing agent, a mutagen, a carcinogen, and radiation. In an embodiment of this method, assessing Tiam1 in contacted cells further comprises determining whether contacting with the agent reverses or prevents senescence.

An aspect of the invention provides a method of using a three-dimensional spheroid cell culture including epithelial cells and fibroblasts in an extracellular matrix, for prognosis of an epithelial cancer, including culturing the epithelial cells and the fibroblasts in the matrix, such that the epithelial cells and the fibroblasts aggregate to form the three-dimensional spheroids, and recovering the spheroids from the matrix and analyzing the epithelial cells for invasiveness or the fibroblasts for gene expression.

In an embodiment of the method, recombinant engineering further comprises modulating expression of at least one gene in at least one of the epithelial cells and the fibroblasts. In an embodiment of the method, the method further includes prior to culturing, recombinant engineering of at least one of the epithelial cells and the fibroblasts. In an embodiment of the method, the method further includes screening a compound for a function reversing the invasiveness of the epithelial cells. In an embodiment of this method, the fibroblasts are human. In an embodiment of this method, the fibroblasts are obtained from a cancer patient tumor biopsy or from a normal subject reduction mammoplasty sample. In an embodiment of the method, the matrix is at least one selected from: BD Matrigel, AlphaMAX 3D, alphaGEL3D, Porocell, BD PuraMatrix, AlgiMatrix, PathClear, Geltrex, MaxGel, HydroMatrix, Mebiol Gel3D, Alvetex, MAPTrix and Cultrex.

In an embodiment of the method, modulating expression of at least one gene further includes modulating expression of Tiam1 or osteospontin in the fibroblasts, such that the method further comprises analyzing invasiveness of the epithelial cells into the extracellular matrix in comparison to control fibroblasts in which gene expression in the fibroblasts is not modulated and the fibroblasts are otherwise identical.

An aspect of the invention provides a method of assessing an agent for modulating invasiveness and metastatic potential of an epithelial cell including contacting a spheroid device comprising fibroblasts and epithelial cells in an extracellular matrix with the agent, and measuring invasiveness of the epithelial cells into the matrix within extent of invasiveness is a measure of the metastatic potential of the epithelial cell.

An aspect of the invention provides a method for evaluating the metastatic potential of an epithelial cell cancer including: detecting Tiam1 in a tissue sample from the epithelial cell cancer and surrounding cells, such that the tissue sample includes fibroblasts and tumor cells or suspected tumor cells, and assessing the Tiam1 expression levels in fibroblasts adjacent to tumor cells, such that a decreased level of Tiam1 expression in the fibroblasts adjacent to tumor cells, compared to level of Tiam1 expression in fibroblasts at a location distant from the tumor, is indicative of increased metastatic potential of the epithelial cell cancer.

In an embodiment of this method, the reagent includes at least one selected from the following group: a small molecule, a protein, an antibody, an enzyme, a nucleic acid, or a nucleic acid. In an embodiment of this method, the fibroblasts are obtained from tissue from the subject.

An aspect of the invention provides a method for modulating metastatic potential of an epithelial cell cancer including contacting fibroblasts associated with an epithelial cell cancer with a reagent that causes increased expression of Tiam1.

An aspect of the invention provides a method for screening a plurality of compounds to identify an agent that modulates metastatic potential of an epithelial cell cancer in a subject including: contacting fibroblasts with at least one compound, and assessing Tiam1 expression levels in resulting contacted fibroblasts, such that increased expression of Tiam1 in the contacted fibroblasts, compared to control fibroblasts not so contacted and otherwise identical, identifies the agent that modulates metastatic potential of the epithelial cell cancer.

An aspect of the invention provides a method of counseling a subject in remission from an epithelial cell cancer, including: obtaining a first biopsy tissue sample from the subject and counseling the subject to avoid exposure to oxidizing agents and to consume anti-oxidant compounds; and, obtaining a second biopsy tissue sample from the subject at a later time, and comparing expression of Tiam1 in fibroblasts in the first tissue sample and the second tissue sample, such that calculating a maintenance of number of fibroblasts expressing high levels of Tiam1 from the time of the first sample to the second sample is an indication of continued remission from the cancer in the subject, and a decreased number of fibroblasts expressing a high level of Tiam1 is an indication of increased risk of recurrence of the cancer.

An aspect of the invention provides a kit for evaluating potential invasiveness of an epithelial cell cancer including: a detection reagent suitable for detecting presence of protein T-cell lymphoma invasion and metastasis-inducing protein 1 (Tiam1) in cells of a tissue sample, such that the tissue sample includes tumor tissue of the cancer and cell tissue surrounding the tumor, and instructions for using the detection reagent to detect Tiam1 in tumor cells of the tissue sample, and in fibroblasts of the tissue sample associated with the tumor, thus evaluating potential invasiveness of the cancer based on amounts of Tiam1 in the tumor cells and in the fibroblasts. In an embodiment of the kit, the instructions include statistical correlations for evaluating the expression of a low amount of Tiam1 in the fibroblasts surrounding the tumor as an indication of a greater likelihood that the tumor is invasive or has increased potential for invasion, metastasis or recurrence, and a high amount of Tiam1 in the fibroblasts surrounding the tumor as an indication that the tumor is non-invasive or has decreased potential for invasion, metastasis or recurrence.

In an embodiment of the kit, the instructions include evaluating the expression of the low amount of Tiam1 in the fibroblasts surrounding the tumor as an indication that the tumor is significantly more likely to be invasive, metastatic or to recur, in view of observations that the Tiam1 expression in the tumor tissue of the cancer is high. In an embodiment of the kit, the kit further contains buffers for deparaffinization and cell conditioning for formalin-fixed paraffin-embedded tissue specimens, and for counterstaining the cells. In an embodiment of the kit, the detection reagent is an antibody that specifically binds to Tiam1. In an embodiment of the kit, the antibody is selected from a polyclonal IgG and a monoclonal antibody.

In an embodiment of the kit, the kit further includes a detection reagent for osteopontin, such that the instructions further comprise evaluating potential invasiveness of the tumor in view of osteopontin levels in the fibroblasts surrounding the tumor. In an embodiment of the kit, the instructions comprise staining tissue from at least one of the group of epithelial cell cancer selected from: breast, prostate, lung, bladder, uterine, ovarian, brain, head and neck, esophageal, pancreatic, gastric, germ cell, and colorectal cancers. In an embodiment of the kit, the instructions include staining tissue from at least one of the group of: fresh biopsy, fresh autopsy, frozen archival, formalin-fixed tissue, alcohol-fixed tissue, paraffin-embedded fixed tissue, a stored slide, and a stored stained slide.

An aspect of the invention provides a kit for evaluating metastatic potential of an epithelial cell cancer including: a detection reagent for detecting an amount of a gene product of a gene encoding T-cell lymphoma invasion and metastasis-inducing protein 1 (Tiam1) in cells of a tissue sample, and instructions for using the detection reagent to detect at least one of: Tiam1 in epithelials cells of the tissue sample; and Tiam1 in fibroblasts of the tissue sample, such that evaluating metastatic potential of epithelial cell cancers is a function of at least one of the amounts of Tiam1 in the epithelial cells of the tissue sample and the fibroblasts in the tissue sample.

In an embodiment of the kit for evaluating metastatic potential, the gene product is Tiam1 protein, and the instructions include methods for immunohistochemical detection. In an embodiment of the kit, the instructions include statistical methods of comparing numbers of Tiam1 positive fibroblasts associated within a specified distance from the cancer with standard numbers of Tiam1 positive fibroblasts from controls including specimens from benign tumors and from known metastatic tumors. In an embodiment of the kit, the detection reagent is an antibody that specifically binds to Tiam1 protein, such that the antibody is a polyclonal antibody or a monoclonal antibody.

### BRIEF DESCRIPTION OF THE DRAWINGS

FIG. 1 panels A-C show a distribution of mammary epithelial cells and fibroblasts in Matrigel co-culture.

FIG. 1 panel A is a photograph of a representative light microscope image from spheroid co-culture established with HMECs and RMFs. Most of the cellular projections extending out from the spheroid are non-fluorescing epithelial cells (arrows).

FIG. 1 panel B is a green fluorescence image of same field. GFP-expressing fibroblasts cluster in the interior core of the spheroid.

FIG. 1 panel C is a hematoxylin and eosin (H&E) staining of a spheroid after ethanol fixation and paraffin embedding. Arrows indicate outgrowths projecting out into the extracellular matrix. Multiple nuclei are seen in each projecting outgrowth.

FIG. 2 is a photograph of a western blot showing levels of Tiam1 (top) and GAPDH (bottom) in the HMEC line and the RMF line. Duplicate lysates are shown for cells transduced with control vector (C) or short hairpin targeting Tiam1 (shTiam). Arrows indicate position of the specific Tiam1 band.

FIG. 3 shows the effect of Tiam1 suppression in mammary fibroblasts on epithelial cell invasion in spheroid co-cultures.

FIG. 3 panel A is a set of photographs of spheroids from co-cultures of HMECs and RMFs with either control (C) or suppressed (sh) levels of Tiam1, that were established in Matrigel in four possible combinations: C-HMEC in 1 and 3; sh HMEC in 2 and 4; C-RMF in 1 and 2; shRMF in 3 and 4. Representative images were taken on day 10. Arrows indicate examples of projections extending out beyond the spheroid perimeter.

FIG. 3 panel B is a bar graph of projection measurements, for which the longest projection on each spheroid from the tip of the projection to the perimeter of the spheroid was measured. Numbers along the X-axis correspond to the co-culture combinations shown in panel A. Data represent mean+/−the standard deviation (S.D.) from 10 representative spheroids in each of 3 separate assays. * indicates p-value <0.0005, ** indicates p-value <0.00005 by two-tailed t-Test.

FIG. 4 is a set of photographs of western blots showing suppression of Tiam1 in human foreskin fibroblasts (HFFs). Western blots of levels of Tiam1 (top panel) and GAPDH (bottom panel) in HFFs were performed with duplicate lysates for parental cells (P), cells transfected panel) in HFFs were performed with duplicate lysates for parental cells (P), cells transfected with control vector (C) or with short hairpin targeting Tiam1 (shTiam). Arrow indicates position of specific Tiam1 band. The data show loss of Tiam1 expression in HFF transfected with shTiam 1.

FIG. 5 shows the effect of Tiam1 suppression in dermal fibroblasts on keratinocyte invasion in 3D human skin equivalents (HSEs).

FIG. 5 panel A is a set of photo micrographic images of human skin equivalents established with either parental HFFs, HFFs with control retroviral vector, or sh-HFFs in the collagen dermis.

FIG. 5 panel B is a graph showing number of projections and number of clusters observed.

FIG. 6 is a line graph of tumor largest dimension (mm) plotted as a function of time (weeks) post injection of the human breast cancer cell line, SUM1315-GFP/luc, after injection into mammary fat pads of NOD-SCID mice. The cells injected were: SUM1315-GFP/luc only (diamonds), or those cells co-mixed with either control RMFs (squares), or shTiam-RMFs (triangles). Each curve represents means+/−S.D. from cohorts of 10 mice.

FIG. 7 is a set of photomicrographs that show the effect of Tiam1 suppression in stromal fibroblasts on breast cancer invasiveness.

Histopathology using H&E was performed on orthotopic tumors from mice implanted with SUM1315-GFP/luc breast cancer cells alone (top panels), or with these cells co-mixed with control RMF (middle panels), or with shTiam1-RMF (bottom panels). Right panels are a 20× magnification of a section from corresponding 10× magnification left panels depicting a representative tumor-stroma interface. T indicates primary tumor, S indicates adjacent stroma. Asterisks indicate murine mammary structures.

FIG. 8 is a set of photomicrographs showing immunohistochemical staining of orthotopic tumors from mice implanted and labeled as shown in FIG. 7. Vimentin staining is shown at 20× magnification. T indicates tumor cells, S indicates adjacent stroma.

FIG. 9 is a set of photomicrographs showing the effect of Tiam1 suppression in stromal fibroblasts on breast cancer metastasis. Histopathology was performed on lung sections from indicate nodular metastatic deposits detectable on H&E staining. Right panels show corresponding vimentin staining at 20× magnification, thin arrows indicate larger metastatic deposits, and asterisks indicate examples of isolated tumor cells detectable only with vimentin staining.

FIG. 10 panels A-C shows the nucleic acid sequence of human Tiam1 mRNA (SEQ ID NO: 2) obtained from GenBank and the predicted amino acid sequence of human Tiam1 protein (SEQ ID NO: 3). The coding region is found at FIG. 10 panel C which shows the amino acid sequence of human Tiam1 protein.

FIG. 11 is a bar graph showing that Rac activation in RMFs depends on Tiam1 expression. Rac activation levels in RMFs with endogenous (control) or suppressed (shTiam) Tiam1 expression grown were determined under quiescent (light gray bars) or pervanadate-stimulated conditions (dark gray bars) as indicated. Data indicate mean+/−S.D. and are representative of duplicate assays, each done in triplicate. The asterisk indicates p-value=0.001 by two-sided t-Test compared with unstimulated control cells.

FIG. 12 is a table and a bar graph showing that suppression of the Rac1 in RMFs does not completely phenocopy the prov-invasive effect of suppression of Tiam1 in RMFs.

FIG. 12 panel A shows results from quantitative real-time PCR for Rac1 and GAPDH in shRac1-RMFs and control shLuciferase-RMFs. Results are shown as mean (S.D) for triplicate samples and are representative of duplicate samples and assays. Rac levels in shRac1-RMFs were observed to be approximately 30% of Rac levels in control cells.

FIG. 12 panel B shows number of spheroids established with HMECs in combination with RMFs with endogenous protein levels (control), suppressed Tiam1 (shTiam), or suppressed Rac1 (shRac), which were visualized by light microscopy. Projections extending beyond spheroid perimeter were counted. Graphs depict spike count distribution for each line. Numbers in parentheses indicate mean projection counts for each line. Differences in mean projection counts were found to be statistically significant between the control and the shTiam (p<0.0001), the control and the shRac (p<0.0013), and the shTiam and the shRac (p<0.0001). An overall test of differences among lines was done using an ANOVA followed by t-tests for pairwise comparisons of cell line means using Sidak adjusted p-values.

FIG. 13 is a set of bar graphs and photographs of western blots that show that osteopontin expression varies inversely with Tiam1 expression in fibroblasts.

Top panels show results of qRT-PCR using osteopontin-specific primers. Data represent mean+/−S.D. for a minimum of three separate samples, each done in triplicate. Bottom panels show immunoblots for osteopontin from concentrated conditioned medium harvested from equal numbers of cells. Samples were loaded in duplicate; numbers below blots indicate quantification by densitometry for triplicate samples. C-RMF indicates RMF with control viral hairpin, shTiam indicates Tiam1-deficient RMF, pBabe indicates RMF with control pBabe vector, and +Tiam indicates Tiam1-over-expressing RMF. For this and subsequent figures, p-values were derived by paired t-Test except as indicated.

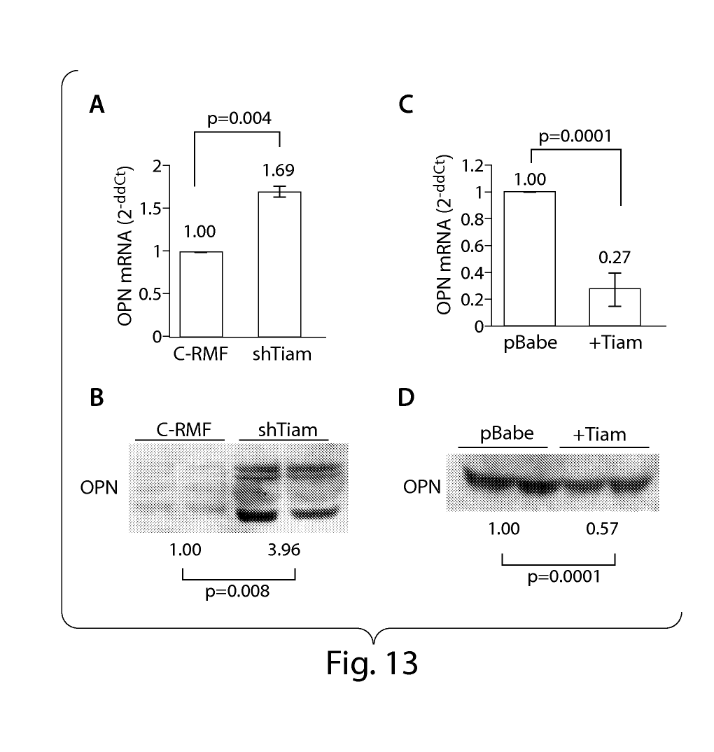

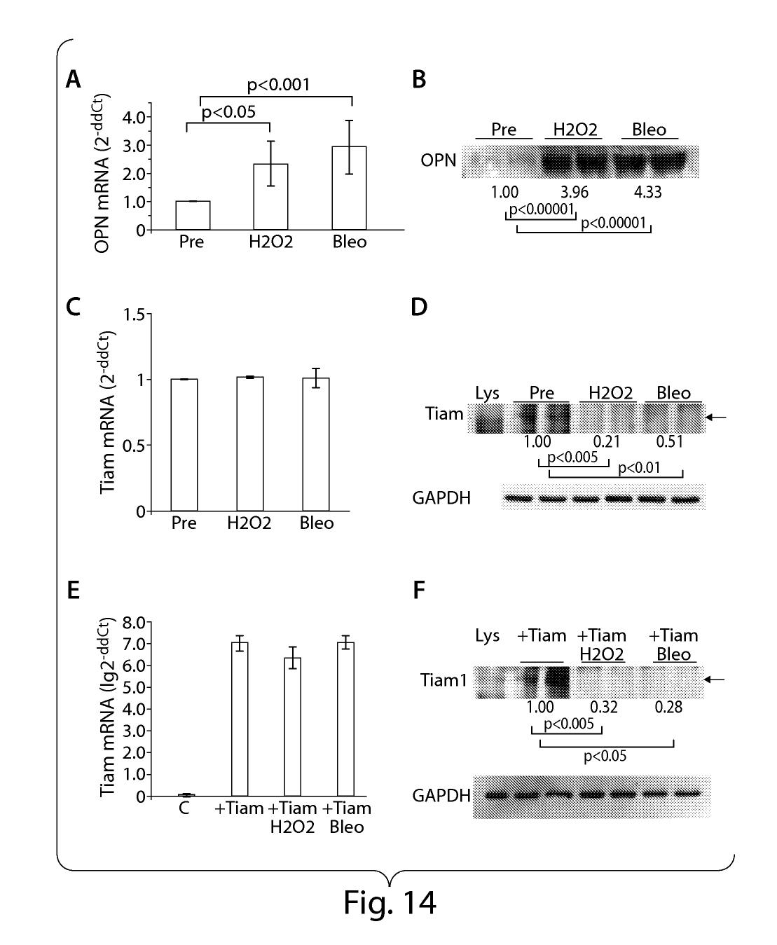

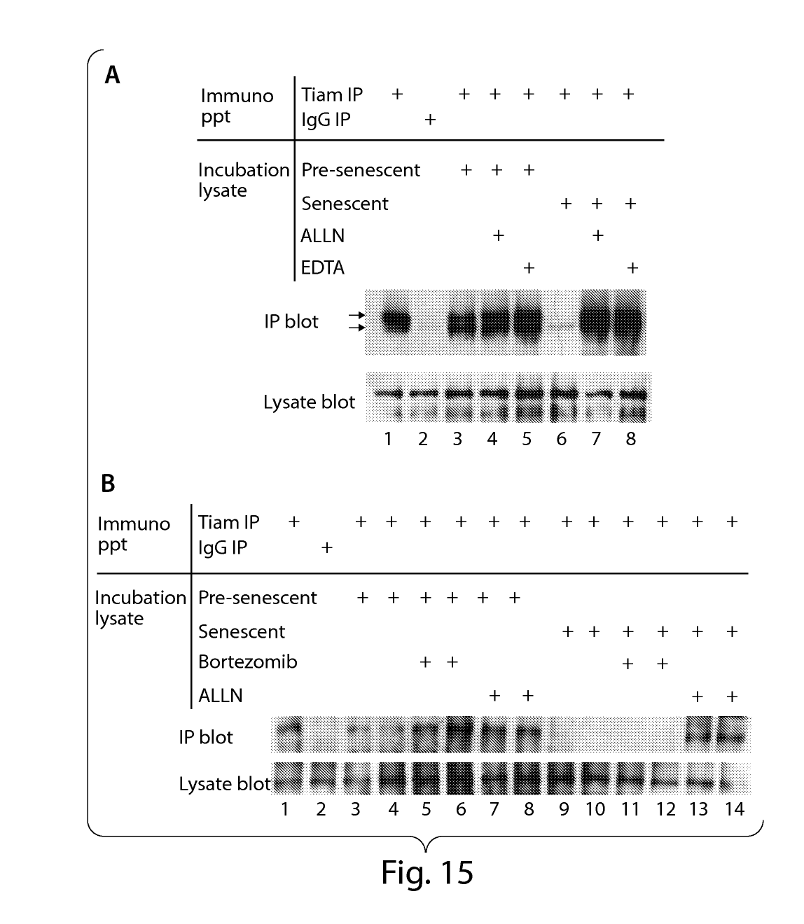

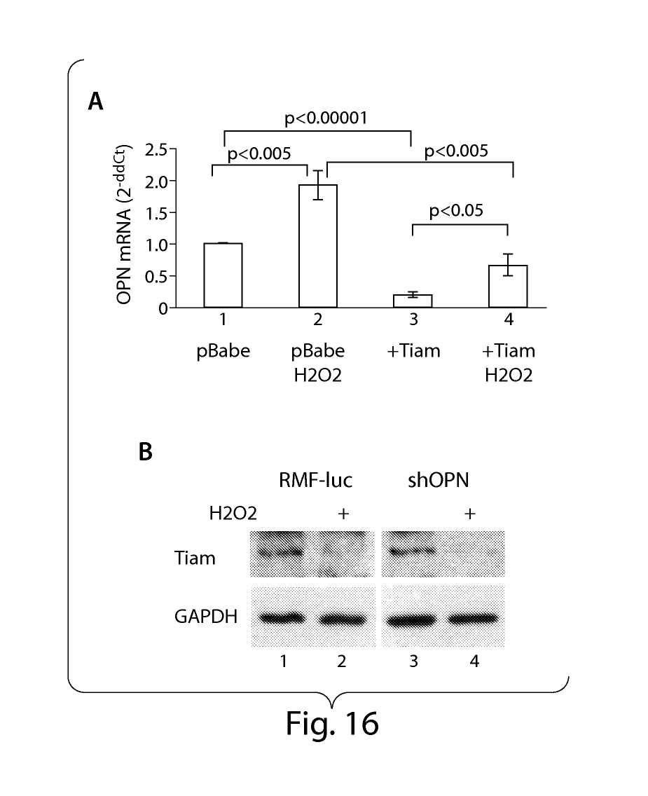

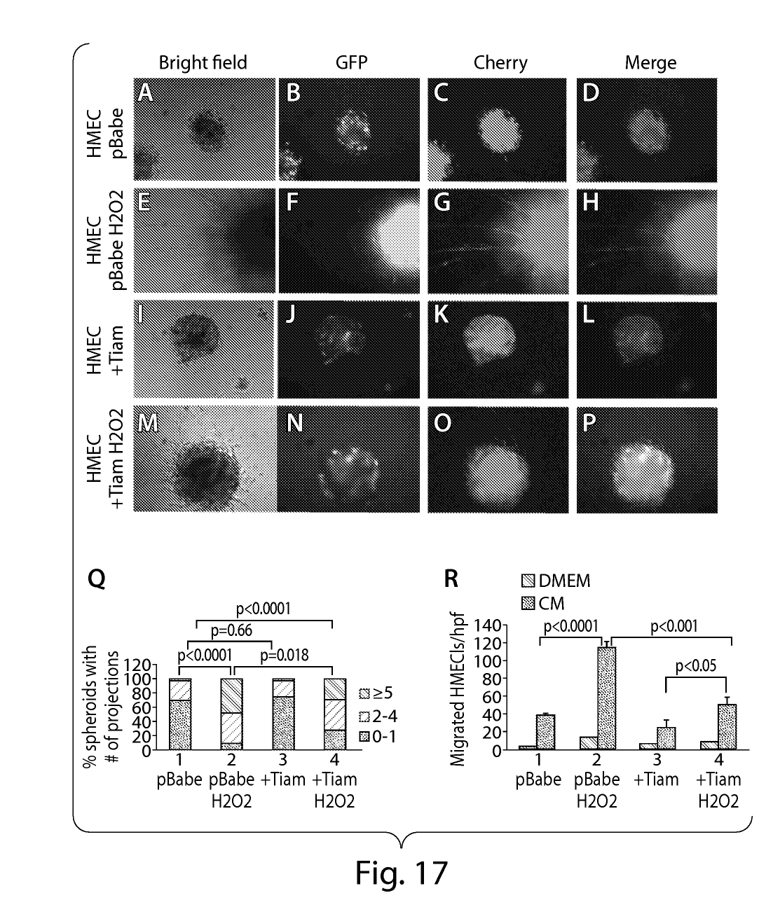

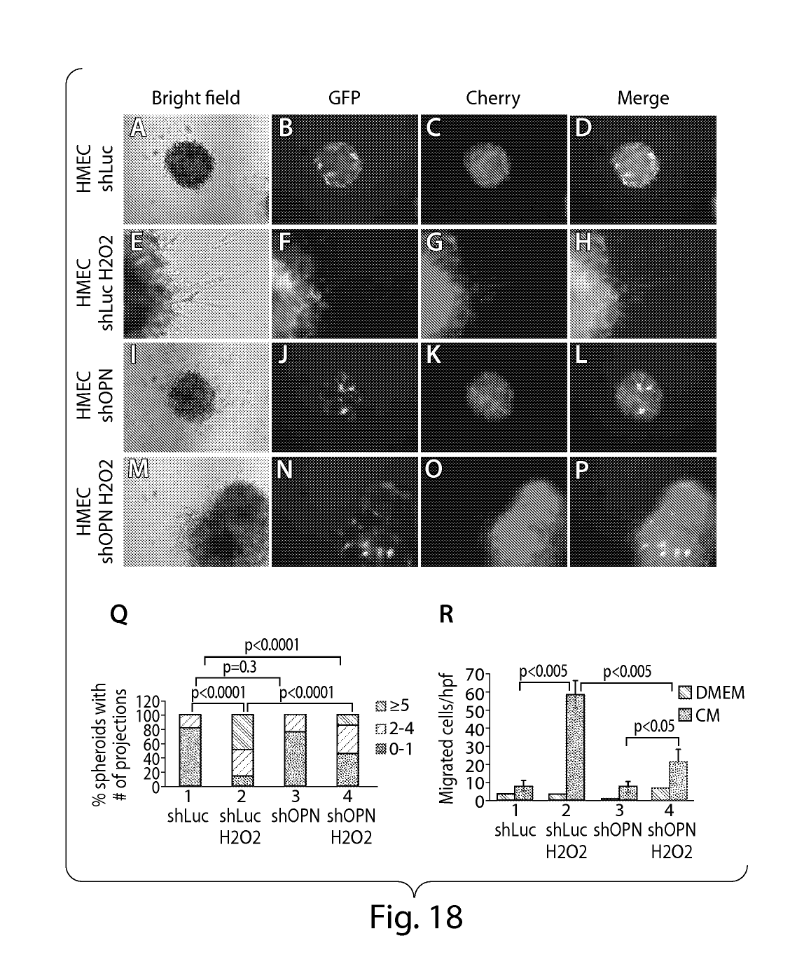

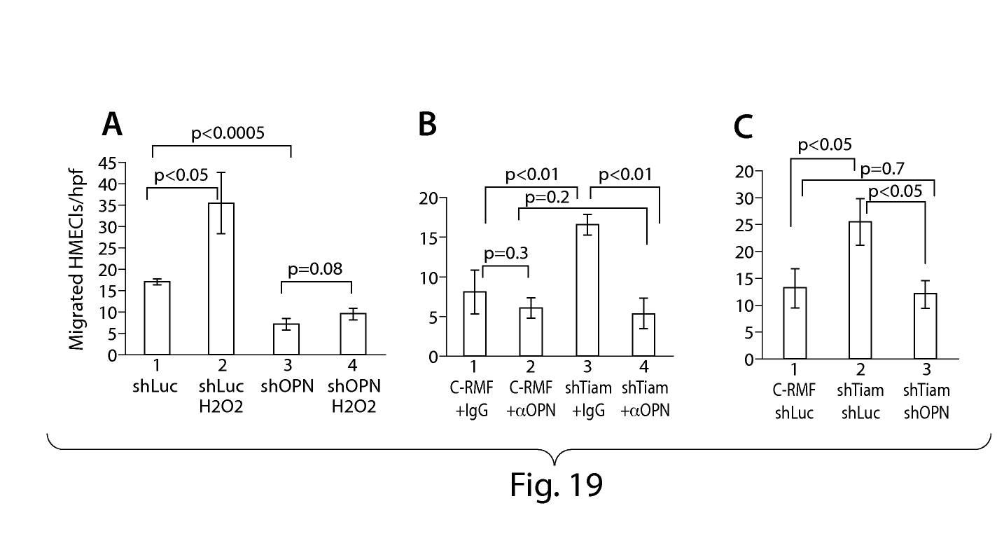

FIG. 14 is a set of graphs and photographs as in FIG. 13 showing that stress-induced senescence leads to inverse changes in osteopontin (OPN) and Tiam1 in fibroblasts.

FIG. 14 panels A, C, E show results of qRT-PCR for OPN or Tiam1 mRNA shown in immunoblots for secreted OPN (B) and Tiam1 or GAPDH as loading control in cell lysates (FIG. 14 panels D and F) as indicated. Results in panels FIG. 14 panels A, C, E indicate mean+/−S.D. for at least three separate assays, each done in triplicate. Results in FIG. 14 panel E are rendered in log scale. Numbers below immunoblots indicate quantification by densitometry for at least three separate assays. Pre indicates pre-senescent cells, H2O2 and Bleo indicate senescence induction with H₂O₂ or bleomycin respectively. In FIG. 14 panels D and F, Lys indicates loading control lysate for position of the full-length Tiam1 band, also indicated by an arrow. In FIG. 14 panel E, C indicates control pBabe RMF, and +Tiam indicates Tiam1-over-expressing RMF.

FIG. 15 is a set of photographs of western blots of immuno-precipitates (IP) or lysates showing that Tiam1 protein is degraded by calpain protease during stress-induced senescence in cells.

FIG. 15 panel A, top shows Tiam1 immunoblot of immunoprecipitates, before or after incubation with lysates from pre-senescent (lanes 3-5) or senescent (lanes 6-8) cells. Incubating lysates was performed with and without pretreatment with calpain inhibitor ALLN or the calcium chelator EDTA as indicated. Arrows indicate position of upper and lower bands of precipitated Tiam1. Results are representative of two independent assays of separate samples.

FIG. 15 panel B shows Tiam1 immunoblot of immunoprecipitates, with and without incubation with lysates from pre-senescent (lanes 3-8) or senescent (lanes 9-14) cells. Lysates were incubated with and without pre-treatment with proteasome inhibitor bortezomib or ALLN as indicated. Results are representative of two independent assays of separate samples. FIG. 15 panels A and B bottom shows Tiam1 immunoblots of lysates from cells with exogenous Tiam1 expression corresponding to IPs above.

FIG. 16 is a bar graph and a photograph showing that Tiam1 regulates OPN expression.

FIG. 16 panel A shows qRT-PCR for OPN mRNA in RMF cells with either endogenous (pBabe) or increased (+Tiam) levels of Tiam1. Results indicate mean+/−S.D. for at least three separate samples, each done in triplicate.

FIG. 16 panel B shows Immunoblots of cell lysates for Tiam1 and GAPDH as indicated from cells with either endogenous (RMF-luc) or deficient (shOPN) levels of OPN. For both panels, H2O2 indicates cells rendered senescent through oxidative stress.

FIG. 17 is a set of photomicrographs and bar graphs showing that up-regulation of Tiam1 in senescent RMF cells inhibits the invasion and migration of associated epithelial cells. FIG. 17 panels A-P show images of co-cultured spheroids in Matrigel taken at 10×. FIG. 17 panels A-D, E-H, I-L, and M-P each show the same field and plane of focus respectively. FIG. 17 panels A-H show co-cultures with HMECs and control pBabe-RMFs. FIG. 17 panels I-P show co-cultures with HMECs and Tiam-overexpressing +Tiam RMFs; panels E-H and M-P show RMF cells rendered senescent prior to establishment of co-cultures.

FIG. 17 panel Q shows numbers of spheroids with specified numbers of HMEC projections per spheroid, expressed as percentage of total spheroids. X-axis indicates specific RMFs in the spheroids. Legend specifies numbers of projections per spheroid. Results are from duplicate separate samples assayed; for each condition at least 25 spheroids were counted in each assay; p-values were determined by Chi-square.

FIG. 17 panel R: results of transwell migration assays on HMECs isolated from co-cultures with RMFs as indicated, expressed as mean+/−S.D. Light (left) and dark (right) blue bars indicate migration toward bottom chamber containing DMEM or DMEM supplemented with conditioned medium respectively.

FIG. 18 is a set of photomicrographs and bar graphs that show that down-regulation of OPN in senescent RMF cells inhibits the invasion and migration of associated epithelial cells.

FIG. 18 panels A-P show representative images of co-cultured spheroids in Matrigel were taken at 10× magnification. FIG. 18 panels A-D, E-H, I-L, and M-P each represent the same field respectively.

FIG. 18 panels A-H show co-cultures with HMECs and control shLuc-RMF cells; panels I-P show co-cultures with HMECs and OPN-deficient (shOPN) RMFs; panels E-H and M-P show RMF cells rendered senescent prior to establishment of co-cultures.

FIG. 18 panel Q shows numbers of spheroids with specified numbers of HMEC projections per spheroid, expressed as percentage of total spheroids. At least 25 spheroids were counted in duplicated assays, legend and statistics were performed as in FIG. 17.

FIG. 18 panel R shows results of transwell migration assays of HMECs isolated from co-cultures with RMFs as indicated, expressed as mean+/−S.D, as in FIG. 17.

FIG. 19 is a set of bar graphs showing that OPN inhibition blocks HMEC migration induced by senescence or Tiam1 deficiency in a seeded cell migration assay.

HMECs were migrated across porous membranes toward bottom chambers containing either FIG. 19 panel A control (shLuc) or OPN-deficient (shOPN) RMFs, or FIG. 19 panel B control or Tiam-deficient (shTiam) RMFs, or FIG. 19 panel C double hairpin control (C-RMF-shLuc), Tiam-deficient RMFs with luciferase hairpin control (shTiam-shLuc), or RMFs deficient in both Tiam1 and OPN (shTiam-shOPN). FIG. 19 panel A shows H₂O₂ indicates RMFs rendered senescent prior to initiation of migration. FIG. 19 panel B shows antibody to OPN (αOPN; bars 2 and 4) or rabbit IgG (IgG; bars 1 and 3) were added in equal amounts to bottom chambers prior to initiation of migration.

### DETAILED DESCRIPTION

There are approximately 250,000 cases of breast cancer diagnosed each year in the United States, of which over 50,000 are non-invasive (DCIS, ductal carcinoma in situ). There has been considerable progress in our understanding of screening and management of breast cancers, with corresponding improvement in survival for women with breast cancer, in particular for those presenting with non-metastatic cancers (the majority of cases). Women with non-invasive breast cancer receive similar treatments as women with node-negative invasive breast cancers—that is, surgical removal of breast tissue, radiation, and often estrogen-blockade therapy—and thus incur the resulting cosmetic deformity, risks and side-effects of these treatments. Women with invasive breast cancers also often receive chemotherapy treatment, and there has been some progress made in adding gene expression array analysis to clinical parameters in order to stratify which tumors would most benefit from chemotherapy and therefore limit the risk of additional side effects. Like invasive breast cancer, DCIS is a heterogeneous disease entity with a range of prognostic outcomes. In contrast to invasive breast cancer, similar prognostic tools have not been demonstrated for DCIS to date. Thus the approach to DCIS combines both under-treatment and over-treatment. A significant fraction of women with DCIS will not incur another breast cancer and are over-treated by the current standard of care. However a sizable fraction (≈20%) will eventually develop recurrent breast cancer, which may be non-invasive, invasive, or even metastatic, and are thus under-treated by the current standard of care. This is of particular relevance for women with high-grade DCIS, who often undergo mastectomy despite the non-invasive nature of their breast cancer, due to heightened concern over their tumor histology. A prognostic tool that better defined risk of recurrent breast cancer in women with DCIS would enable more appropriate therapeutic decision-making, better clinical trial design for potential therapeutic interventions, minimize toxicity, and maximize efficacy.

Most of the effort in developing prognostic tools in cancers has focused on the tumors themselves. However, the role of the microenvironment in tumor development is gaining increased recognition. There is growing evidence that the stromal microenvironment around cancer cells influences the growth, invasiveness, and metastatic behavior of cancer cells, and perhaps also therapeutic response. It is becoming increasingly apparent that there are bidirectional signals (co-evolution) between cancer cells and tumor-associated stroma. Tumor-associated stroma is comprised of various cell types and panoply of extracellular molecules comprising or secreted into the extracellular matrix. The list of factors that participate in the co-evolution of tumors with tumor-associated stroma is growing, and the interplay between signaling pathways within the tumor cells and the stroma itself is beginning to be understood. Of note, fibroblasts are the predominant cell type in stromal connective tissue, contributing to deposition and maintenance of basement membrane and paracrine growth factors. There is a need to determine a diagnosis from a fibroblast actively function in the induction of cancers.

The Rac exchange factor Tiam1 is shown in Examples herein to have important role in the microenvironment around human breast cancers with regard to regulation of tumor invasion and metastasis. These data resolve a major paradox in the current understanding of Tiam1. Tiam1 is a ubiquitous protein and its expression in tumor cells seems to be required for facilitating tumor growth. However, the tumors that do develop in Tiam1 knock-out mice are more invasive, conceptually inconsistent with the requirement for Tiam1 for tumor growth and also inconsistent with the behavior of human tumors clinically. We therefore hypothesized that in these mice, in which Tiam1 knock-out is neither conditional nor tissue-specific, decreased tumor growth is due to Tiam1 deficiency in the tumor cells themselves, while the increased tumor invasion is due to Tiam1 deficiency in the fibroblasts of the tumor stroma. We tested this hypothesis in 3 different systems, including three-dimensional mixed cell spheroid cultures of mammary epithelial and fibroblast cells, a dimensional organotypic culture model of human skin, and a mouse model of human breast cancer. In all three models, Tiam1 deficiency in human fibroblasts (engineered using retroviral delivery of short hairpin DNAs based on short interfering RNA templates targeting Tiam1) led to increased invasiveness of the epithelial and tumor cells being studied. In the mouse model, Tiam1 deficiency in the fibroblasts was also associated with increased metastasis of the tumors. These results support our hypothesis that Tiam1 silencing in stromal fibroblasts promotes tumor cell invasion and metastasis. This new function for Tiam1 in the tumor microenvironment is a highly novel finding, as prior work on Tiam1 related to cancer had focused on the role of Tiam1 in the cells of the cancer.

Tiam1 expression in breast cancer-associated fibroblasts is relevant to human disease and not just experimental models as shown herein. In an immunohistochemical study using a commercially available polyclonal anti-Tiam1 antibody (Santa Cruz), no fibroblasts immediately adjacent to invasive breast cancers were found to express detectable Tiam1. In contrast, in most cases of high-grade DCIS the immediately adjacent fibroblasts displayed high expression of Tiam1. The difference between Tiam1 expression in invasive vs non-invasive cancers was highly statistically significant [p<0.001; 95% confidence interval for the difference of (0.60, 0.97)]. The range of follow-up for these cases was quite variable (3 months to 11 years), but of note one of the cases of DCIS without fibroblast Tiam1 expression at the time of initial diagnosis has since suffered a relapse with invasive, node-positive breast cancer 7 years later. Thus Tiam1 expression in breast cancer-associated fibroblasts is inversely correlated with invasive potential in human breast cancers, confirming relevance to human disease. More specifically, our results suggest that Tiam1 expression in fibroblasts associated with non-invasive breast cancers may be useful as a biomarker for prognosis of future biologic behavior.

We propose that expression of Tiam1 in fibroblasts associated with high-grade DCIS is a marker of favorable clinical outcome (no recurrence), while lack of expression of Tiam1 in fibroblasts associated with high-grade DCIS is a marker of increased risk of unfavorable clinical outcome (recurrence). Therefore an antibody with high specificity for detecting Tiam1 in pathologic samples would be a useful prognostic biomarker in this disease. There are several anti-Tiam1 antibodies currently available for scientific use, but only one that works for immunohistochemistry of formalin-fixed tissues, and it lacks specificity, as demonstrated by the detection of multiple non-specific bands on immunoblot. Thus we propose development of a monoclonal anti-Tiam1 antibody specifically optimized for detection of Tiam1 in formalin-fixed, paraffin-embedded human tissue samples.

Methods and kits for evaluating cancers are provided herein. The methods and kits provided herein are used in the evaluation of carcinomas that is cancers that originate in epithelial cells. In some embodiments, the methods and kits provided herein can involve or be used to detect or modulate Tiam1 expression. In other embodiments, the methods and kits provided herein are used to screen for compounds that modulate Tiam1 expression.

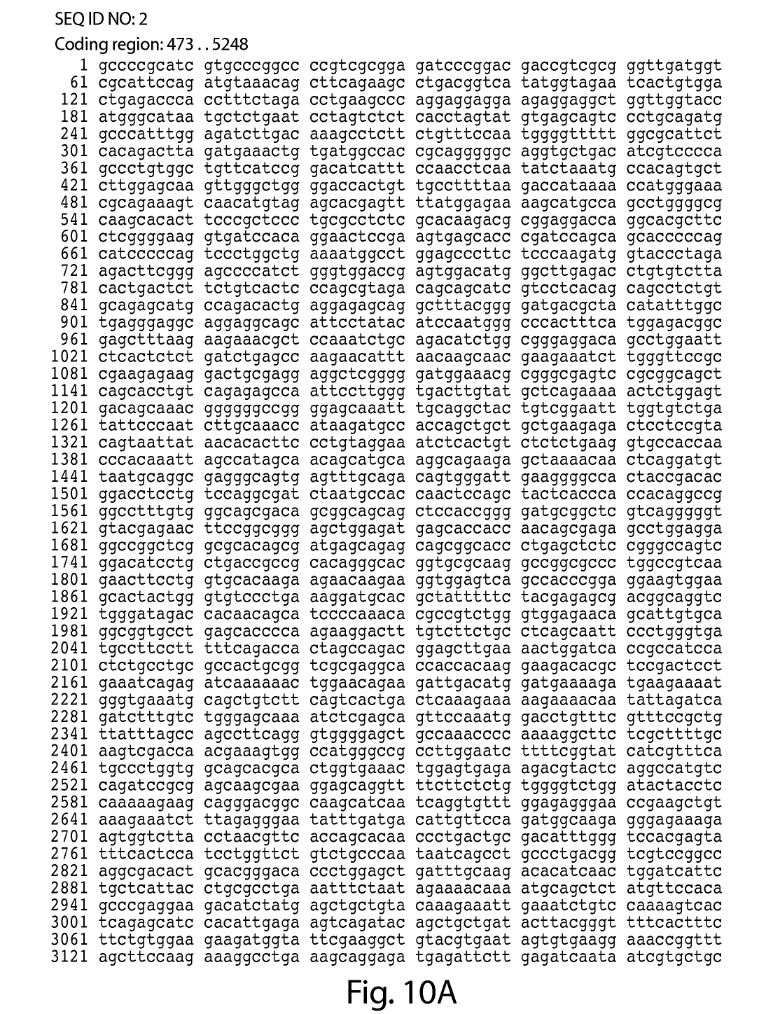

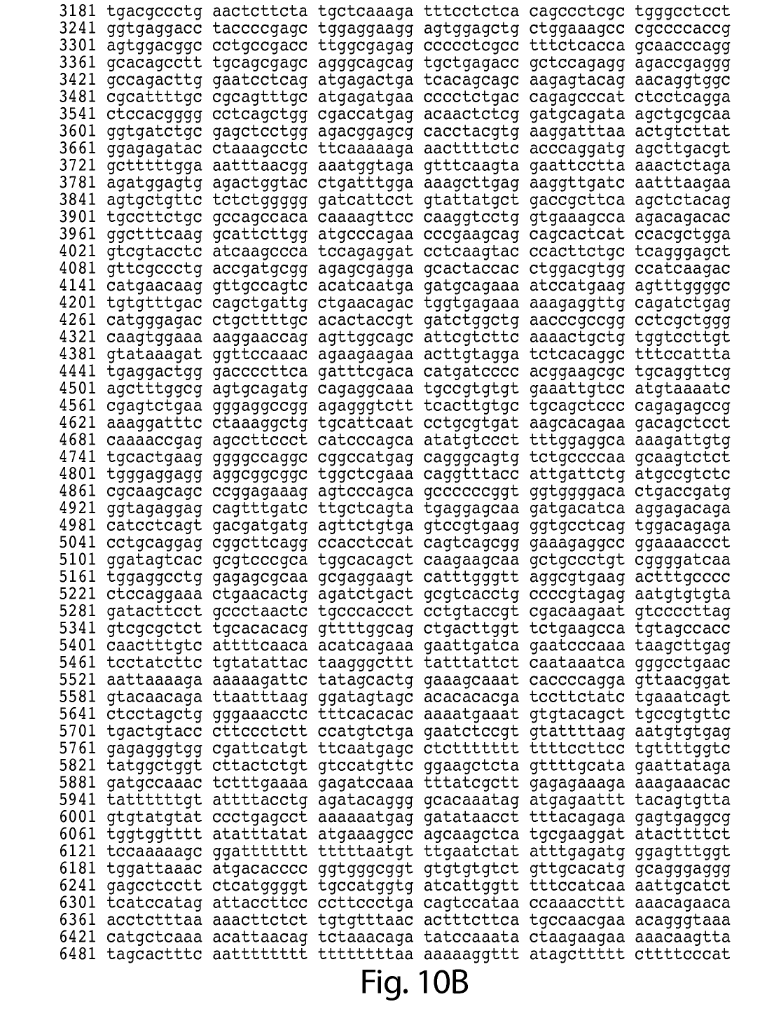

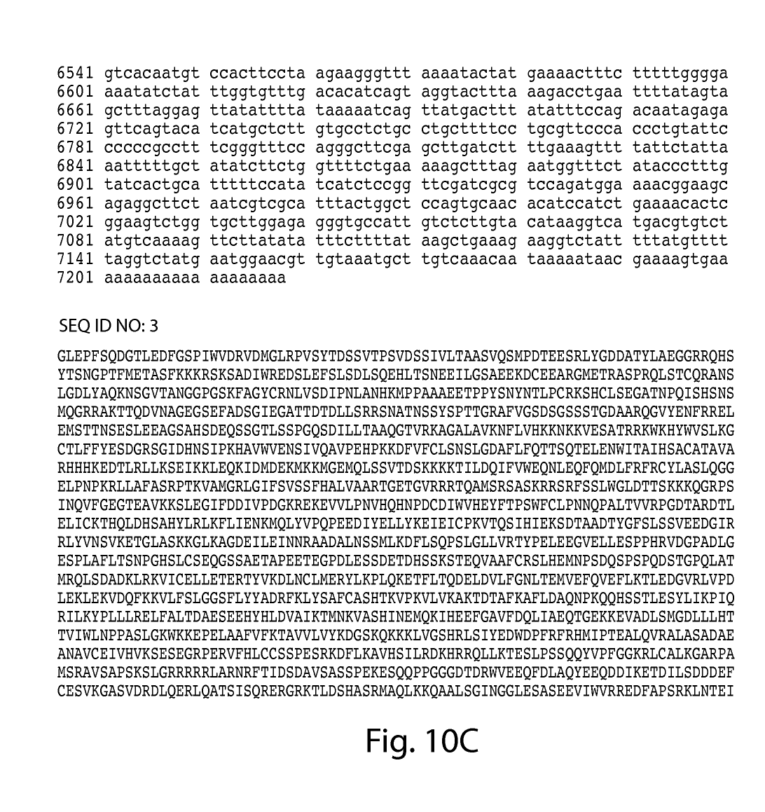

Tiam1 is a protein expressed in human cells, and the nucleic acid and amino acid sequences are shown in FIG. 10A-C. Suitable homologs and alleles of Tiam1 can also be used in the technology provided herein. Suitable homologs and or alleles will have similar activity compared to Tiam1 and are identified by conventional techniques. For example, a homolog to Tiam1 polypeptide is a polypeptide from a human or other animal that has a high degree of structural similarity to Tiam1. In some embodiments, a suitable Tiam1 protein homolog has at least 95% percent identity in amino acid sequence compared to Tiam1. In some embodiments, a suitable Tiam1 gene homolog has at least 90% percent identity in nucleic acid sequence compared to Tiam1. In some embodiments, suitable homologs share at least 95% nucleotide identity and/or at least 97% amino acid identity with Tiam1. In some embodiments, suitable homologs share at least 97% nucleotide identity and/or at least 99% amino acid identity with Tiam1. The homology is calculated using publicly available software tools developed by NCBI (Bethesda, Md.). The software is obtained through the internet. Exemplary tools include the BLAST system available from the website of the National Center for Biotechnology Information (NCBI) at the National Institutes of Health. Pairwise and ClustalW alignments (BLOSUM30 matrix setting) as well as Kyte-Doolittle hydropathic analysis are obtained using the MacVector sequence analysis software (Oxford Molecular Group).

A tissue sample obtained from an individual is used in some embodiments of the methods and kits provided herein. In some embodiments, the individual has or is suspected of having cancer. The individual suspected of having cancer such as breast cancer may be identified, for example, by manual examination, biopsy, family medical history, the subject's medical history, or based on one or more imaging techniques known to those skilled in the medical arts. In some embodiments, the individual from whom the tissue sample is obtained is at risk for developing cancer. Individuals at risk for developing cancer are identified, for example, based on family medical history and/or based on genetic testing of one or more genes known or suspected of being involved in cancer.

The methods and kits provided herein are used to evaluate potential invasiveness and/or metastatic potential of a cancer. Suitable cancers that are evaluated using the technology provided herein include carcinomas (cancers of epithelial cells). Suitable carcinomas include squamous cell carcinoma, adenocarcinoma, and transitional cell carcinoma. The carcinoma is a ductal carcinoma or a non-ductal carcinoma. The squamous cell carcinoma is for example, from the skin, lips, mouth, esophagus, urinary bladder, prostate, lungs, vagina, or cervix. The adenocarcinoma is for example, from the colon, lung, cervix, prostate, urachus, vagina, breast, esophagus, pancreas, or stomach. In some embodiments, the epithelial cell cancer is breast cancer. In some embodiments, the breast cancer is a ductal carcinoma in situ (DCIS), in other embodiments, the breast cancer is a lobular carcinoma.

One or more tissue samples is used in the methods and kits provided herein. Any tissue sample that includes or is suspected to include cancer cells and fibroblasts adjacent to the cancer cells or suspected cancer cells is used with the technology provided herein. In some embodiments, fibroblasts adjacent to cancer cells (or suspected cancer cells) are those that are in physical contact or at least partial physical contact with one or more cancer cells or suspected cancer cells. In some embodiments, fibroblasts adjacent to cancer cells or suspected cancer cells are located a few cells away from the cancer cells. A few cells includes, for example, one, two, three, four, five or more cells.

In some embodiments, the tissue sample comprises a collection of cells obtained from an individual. The tissue sample is obtained from an individual by a conventional means that allows detection of Tiam1 expression levels in cells or cell fragments of the sample. The tissue sample is obtained from a tumor (or suspected tumor) and/or from tissue near the tumor or from a suspected tumor, for example, within the same organ or region of the organ or is obtained from an area that is of the same tissue type but more removed from the tumor. The source of the tissue sample is solid tissue such as muscle or organ tissue. The tissue sample is for example a portion of the tissue originally obtained from the individual. In some embodiments, the tissue sample comprises blood or blood constituents; bodily fluids such as cerebral spinal fluid, amniotic fluid, peritoneal fluid, or interstitial fluid; cells from any time in gestation or development of the subject or individual. Any tissue sample from a subject may be used. Examples of tissue samples include, but are not limited to, breast, prostate, ovary, colon, lung, endometrium, stomach, salivary gland or pancreas depending on the type of cancer or suspected cancer being evaluated. In some embodiments, the tissue sample includes tumor cells such as cells associated with the tumor or found in the tumor microenvironment. For example, cells associated with the tumor include fibroblasts such as stromal fibroblasts. The tissue sample may contain compounds not naturally intermixed with the tissue in nature such as preservatives, anticoagulants, buffers, fixatives, nutrients, antibiotics, or the like. The tissue sample is obtained from certain embodiments from primary or cultured cells or cell lines.

The tissue sample is obtained by any procedure including, but not limited to surgical excision, aspiration, or biopsy. The tissue sample is fresh, frozen and/or preserved, for example, the tissue sample is fixed and embedded in paraffin or the like.

In some embodiments, the tissue sample is divided into pieces or is sectioned prior to or after use with the kits or in the methods provided herein. A “section” of a tissue sample is, for example, a thin slice of the tissue sample or cells microtomed or cut from a tissue sample.

The tissue sample is fixed (or preserved) by conventional methodology [Manual of Histological Staining Method of the Armed Forces Institute of Pathology, 3re edition (1960) Lee G. Luna, H T (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C]. One of skill in the art will appreciate that the choice of a fixative is determined by the purpose for which the tissue is to be histologically stained or otherwise analyzed. One of skill in the art will also appreciate that the length of fixation depends upon the size of the tissue sample and the fixative used. By way of example, neutral buffered formalin, Bouin's or paraformaldehyde, is used to fix a tissue sample. Generally, the tissue sample is first fixed and is then dehydrated through an ascending series of concentrations of alcohols, infiltrated and embedded with paraffin or other sectioning media so that the tissue sample is sectioned. Alternatively, the tissue is sectioned and the sections are fixed. By way of example, the tissue sample is embedded and processed in paraffin by conventional methodology. Once the tissue sample is embedded, the sample may be sectioned by a microtome. By way of example for this procedure, sections may range from about three microns to about five microns in thickness. Once sectioned, the sections may be attached to slides by several standard methods. Examples of slide adhesives include, but are not limited to, silane, gelatin, poly-L-lysine and the like. By way of example, the paraffin embedded sections may be attached to positively charged slides and/or slides coated with poly-L-lysine.

If paraffin has been used as the embedding material, the tissue sections are deparaffinized and rehydrated with water. The tissue sections may be deparaffinized by several conventional standard methodologies. For example, xylenes and a gradually descending series of alcohols may be used [Ibid.]. Alternatively, commercially available deparaffinizing agents are used.

In some embodiments, the tissue sample or sections thereof are mounted on slides and are stained with a morphological stain for evaluation. Any suitable morphological stain is used. A suitable morphological stain is one that provides for accurate morphological evaluation of the tissue sample or section thereof and also allows for accurate assessment of the level of Tiam1 detected. After staining, the tissue sample or section thereof is analyzed by standard techniques of microscopy. Generally, a pathologist or the like assesses the tissue for the presence of abnormal or normal cells or a specific cell type and provides the loci of the cell types of interest. Suitable morphological stains include, for example, hematoxylin and eosin.

Any means of defining the loci of the cells of interest may be used (for example, coordinates on an X-Y axis). In some embodiments, after light microscopy and prior to evaluating the level of expression of Tiam1, the slides are destained by conventional methodology. In some embodiments, the slides are not destained prior to evaluating the level of expression of Tiam1.

In some embodiments, Tiam1 expression level is determined using immunohistochemistry (IHC). In some embodiments, IHC is performed in combination with morphological staining as described herein.

In some embodiments, direct IHC is performed. In direct IHC, binding of antibody to the target antigen (for example, Tiam1) is determined directly using a labeled antibody that is capable of binding the target antigen. In other embodiments, indirect IHC is used. In indirect IHC, an unlabeled primary antibody that is capable of binding the target antigen is used followed by a labeled secondary antibody that is capable of binding to the primary antibody. Where the secondary antibody is conjugated to an enzymatic label, a chromagenic or fluorogenic substrate is added to provide visualization of the antigen.

The primary and/or secondary antibody used for immunohistochemistry typically in some embodiments is labeled with a detectable moiety. Numerous labels are available such as: radioisotopes, fluorescent molecules, and enzymatic reagents.

Suitable radioisotopes include, for example, ³⁵S, ¹⁴C, ¹²⁵I, ³H, and ¹³¹I. The antibody is labeled with the radioisotope using known techniques [Coligen, et al., 1991, Current Protocols in Immunology Volumes 1 and 2, Ed]. Other suitable labels include, for example, colloidal gold particles, fluorescent labels such as rare earth chelates (europium chelates), Texas Red, rhodamine, fluorescein, dansyl, Lissamine, umbelliferone, phycocrytherin, phycocyanin, SPECTRUM ORANGE® and SPECTRUM GREEN® and/or derivatives of any one or more of the above. The fluorescent labels are conjugated to the antibody using known techniques [Coligen, et al., 1991, Current Protocols in Immunology, Volumes 1 and 2, Ed]. Suitable enzyme-substrate labels are available and described in U.S. Pat. No. 4,275,149. The enzyme generally catalyzes a chemical alteration of the chromogenic substrate that is measured using various techniques. For example, the enzyme may catalyze a color change in a substrate, which is measured spectrophotometrically. Alternatively, the enzyme alters the fluorescence or chemiluminescence of the substrate.

A chemiluminescent substrate becomes electronically excited by a chemical reaction and may then emit light, which is measured (using a chemiluminometer, for example) or donates energy to a fluorescent acceptor. Examples of enzymatic labels include luciferases (e.g. firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Suitable labeled and unlabeled secondary antibodies are available commercially and techniques for conjugating enzymes to antibodies are known in the art [O'Sullivan M et al. 1981, Methods in Enzym. (ed J. Langone & H. Van Vunakis), Academic press, New York, 73:147-166].

In some embodiments, the label is indirectly conjugated to the antibody. For example, the antibody is conjugated with biotin and any of the four broad categories of labels mentioned above is conjugated with avidin, or vice versa. Biotin binds selectively to avidin and thus, the label is conjugated with the antibody in this indirect manner. Alternatively, to achieve indirect conjugation of the label with the antibody, the antibody is conjugated with a small hapten and one of the different types of labels mentioned above is conjugated with an anti-hapten antibody. Thus, indirect conjugation of the label with the antibody is achieved.

In addition to the sample preparation procedures described herein, further treatment of the tissue section prior to, during or following IHC may be desired. For example, epitope retrieval methods, such as heating the tissue sample in citrate buffer, are performed [Leong, et al., 1996, Appl. Immunohistochem 4:201]. Following an optional blocking step, the tissue sample or section thereof is exposed to primary antibody for a sufficient period of time and under suitable conditions such that the primary antibody binds specifically to the target protein antigen in the tissue sample or section thereof. Appropriate conditions for achieving specific binding are well known in the art and are determined by routine analysis. The primary antibody in some embodiments is detectably labeled, and the extent of binding of antibody to the sample is determined. Alternatively the primary antibody is not detectably labeled, and the tissue sample is exposed to a reagent that allows detection of the primary antibody bound to the target antigen, such as a secondary antibody described herein.

In some embodiments, the tissue sample or section thereof is mounted on a slide (before or after staining and/or subjected to protein antigen detection as described above) and a coverslip is applied. Evaluation and scoring of the tissue samples is performed as described herein.

Expression level of Tiam1 is assessed in a quantitative or qualitative manner. Quantitative evaluation is accomplished, for example, by manual or automated scoring of a tissue sample that has been treated to reveal the presence of Tiam1 (by IHC, for example). The amount of detectable label present is adjusted to remove background label and compared to a control. The tissue sample Tiam1 expression level is expressed as a percentage of the expression level of the control. In some embodiments, the scoring is qualitative, for example, expressed as more than or less than the level of the control. The degree of increased or decreased expression is denoted by +1, +2, +3, for example, with +3 being the highest level of expression seen or expected to be seen compared to the control or −1, −2, −3, for example, with −3 being the lowest level of expression seen or expected to be seen compared to a control. As described herein, decreased expression is any amount of expression that is lower than that seen or expected in a control. Decreased expression is for example more than 1% less, more than 5% less, more than 10% less, more than 25% less, more than 50% less, more than 75% less, more than 90% less or more than 95% less expression compared to a control.

In some embodiments, the methods provided herein comprise comparing the expression of Tiam1 in a tissue sample to a control. The control is any suitable sample that allows a comparative assessment of the level of Tiam1 expression in the tissue sample being tested. The control is a tissue sample obtained from the same individual or is obtained from a different individual. The tissue for the control is obtained from an individual that is not suspected of having cancer or cancer of that particular tissue type. The control is from the same tissue type as the tissue sample being evaluated or can be from a different tissue type. The control is cells from cell culture or cells from a three-dimensional tissue culture model. To obtain the standard image, the control is processed in the same or similar manner as the tissue sample. The control is standard image of normal tissue or cell culture sample. Suitable tissue for the control includes any tissue that is likely to have normal or baseline Tiam1 expression levels (that is, Tiam1 expression levels that are unaffected by cancer).

Kits suitable for use in the methods described herein are provided. In some embodiments, the kits are suitable for evaluating potential invasiveness of an epithelial cell cancer. In another embodiment, kits suitable for evaluating metastatic potential of an epithelial cell cancer are provided. In another embodiment, the kits are suitable for evaluating potential invasiveness and/or metastatic potential of an epithelial cell cancer. In some embodiments, the kits include reagents suitable for detecting Tiam1 expression levels in cells. In other embodiments, the kits include instructions.

In some embodiments, the tissue sample is obtained from the individual and used with the kits or in the methods provided herein by the medical professional (such as a doctor, nurse, or technician) or the medical institution that is treating or evaluating the individual. In other embodiments, the tissue sample is obtained by the medical professional and sent to another facility or service to be used with the kits or in the methods provided herein. As used herein, an evaluator is a person or machine that uses the kits and or methods provided herein to evaluate a tissue sample. The evaluator can be, for example, a pathologist. In some embodiments, at least one of the method steps is performed by a machine.

The kits provided herein can include one or more detection reagents suitable for detecting Tiam1 expression levels in cells of a tissue sample. In some embodiments, the instructions describe how to use the detection reagent to detect Tiam1 expression levels cells of a tissue sample. Suitable detection reagents are those that permit detection of Tiam1, for example by IHC. Suitable detection reagents can also include one or more counter stains.

Other suitable methods of detecting Tiam1 expression in tissues include: in situ hybridization of tissues and/or cells using Tiam1-specific nucleic acid probes, qRT-PCR (quantitative real-time polymerase chain reaction) of RNA from tissue samples obtained by laser-capture micro dissection, and immunoblot (Western blot) of lysates of cells or tissues. Methods for in situ hybridization of tissues and/or cells using nucleic acid probes are described, in Singer et al. U.S. Pat. No. 4,888,278 “In-situ hybridization to detect nucleic acid sequences in morphologically intact cells,” to Methods for qRT-PCR are described, for example, in O'Hagan et al. U.S. Pat. No. 7,544,476 “Identifying cancers sensitive to treatment with inhibitors of notch signaling,” Methods for Western blot analysis of lysates of cells or tissues are described, in Antibodies, A Laboratory Manual, Harlow and Lane, Cold Spring Harbor Laboratory Press, 1988.

In some embodiments, the instructions describe how to assess Tiam1 expression levels in the tissue sample using the one or more detection reagents. In some embodiments, the instructions describe how to evaluate and potential invasiveness of the tumor based on the Tiam1 expression levels. In some embodiments, the instructions describe how to evaluate metastatic potential of the tumor based on the Tiam1 expression levels.

In some embodiments, Tiam1 expression levels in cells of the tissue sample are evaluated or determined. In one embodiment, decreased level of Tiam1 expression in at least some of the cells of the sample is indicative of increased potential invasiveness the cancer in the individual. The cells are, for example, cells adjacent to the tumor or suspected tumor. The cells associated with the tumor can be, for example, fibroblasts such as stromal fibroblasts.

Methods for modulating cancer progression and/or preventing cancer recurrence are provided. In some embodiments, methods for modulating potential invasiveness of the cancer are provided. In some embodiments, methods of modulating metastatic potential of a cancer are provided. In some embodiments, methods of modulating the cancer comprise contacting cells associated with the cancer with a reagent that causes increased expression of Tiam1. Cells associated with the cancer are, for example, fibroblasts. In one embodiment, the fibroblasts are stromal fibroblasts. In another embodiment, the fibroblasts are adjacent to the tumor.

Methods of screening compounds for those compounds that may modulate a cancer are provided. In some embodiments, compounds are screened for those that modulate potential invasiveness of a cancer. In some embodiments, compounds are screened for those that modulate metastatic potential of a cancer. In some embodiments, the method of screening comprises contacting cells associated with the epithelial cell cancer with one or more candidate compounds. In some embodiments, the method of screening comprises assessing Tiam1 expression levels in the contacted cells. In some embodiments, increased expression of Tiam1 in the contacted cells is indicative of a compound that may modulate invasiveness of the cancer. In some embodiments, increased expression of Tiam1 in the contacted cells is indicative of a compound that may modulate metastatic potential of the cancer. Cells associated with cancer are described above.

As described herein, contacting includes exposing in cell culture to the compound. Cell culture includes traditional mammalian cell culture as well as 3D cell culture models. Suitable 3D cell culture models include the use of collagen gels, sponges, and composites, extracellular matrix extracts, collagen/ECM composites, tissue matrix scaffolds, fibrin and composites, crosslinked glycosaminoglycan composites, silk composites, alginates, hydrogels and composites, synthetic scaffolds, nanofibers and peptide scaffolds, and chitosan composites [Yamada K M, et al., 2007, Cell 130:601-610].

Contacting also includes contacting cells in an individual by administering a given compound. Suitable modes of administering a compound to an individual include modes that allow the compound to reach the cell upon administration, for example by topical administration or direct injection at or near the location of the cells to be contacted. Topical administration includes application to the skin, or alimentary canal lining (for example by ingesting the compound or by suppository), or to genitourinary, pulmonary, nasal, and ophthalmic lining. Suitable modes of administering a compound also include modes that allow the compound to diffuse to the cell, such as through the blood or lymph, through the epidermis, or through genitourinary, pulmonary, nasal, and ophthalmic routes, or through the lining of the alimentary canal. A suitable mode of administering the compound is one that allows a sufficient quantity or concentration of the compound to contact the cell to allow a measurable effect on Tiam1 expression. The effect on Tiam1 expression is long (24 hours or more) or short (less than 24 hours).

### EXAMPLES

The following materials and methods are used throughout the Examples herein.

### Example 1

### Cell Culture

H-TERT immortalized human mammary epithelial cells (HMECs) were cultured in DME/F12 medium (HyClone) enriched with 5% bovine calf serum, 5 μg/mL insulin, 1 μg/mL hydrocortisone, and 10 ng/mL EGF. H-TERT immortalized reduction mammary fibroblasts (RMFs) were grown in Dulbecco's modified Eagle's medium (DMEM) containing 10% bovine calf serum. HEK293T cells were grown in DMEM supplemented with 10% iron-supplemented bovine calf serum (Hyclone). 293FT cells for lentivirus production were grown in DMEM supplemented with 10% fetal bovine serum, 0.1 mM MEM Non-essential amino acids, and 2 mM L-glutamine Mouse embryo fibroblasts (MEFs) were cultured in DMEM supplemented with 10% fetal bovine serum. All culture media contained 100 units/ml penicillin, 100 μg/mL of streptomycin and 0.1% fungizone. Cells were cultured in an incubator with humidified air (5% CO2) at 37° C. in plastic dishes or otherwise as described.

For collection of RMF-conditioned medium for protein assay, RMF cells were plated at a density of 3.0×10⁵ per 100-mm dish. Cells reached 80% confluence approximately 24 hours after being seeded, at which point the medium was replaced with serum-free medium. Conditioned medium was collected 24 hours later, concentrated 10× using VIVASPIN 20 (Sartorius Stedium, 3,000 MWCO PES), and stored at −20° C. until use.

### Example 2

### Rac Activation Assay

Rac activation in RMF cell lines was assessed using an ELISA-based assay with colorimetric read-out (Rac G-LISA Activation Assay kit; Cytoskeleton, Inc) according to manufacturer's instructions. Cells were serum-deprived for 16 hours; then some were stimulated with 200 μM pervanadate for 10 minutes. Assays were carried out in 96-well plates; signals were detected by absorbance at 490 nm using a SpectraMax 340 microplate spectrophotometer.

### Example 3

### Spheroid Co-Culture in Matrigel

Matrigel (BD Biosciences) was diluted in 1:1 ratio with ice-cold HMEC medium, and 30 μL were placed mid-well in a 24-well plate. After incubating for 5 min in 37° C., an additional 200 μL of Matrigel:medium mixture was added into the well and incubated for another 30 min A 1:1 mixture of HMEC and RMF cells (0.75×10⁵ cells each) in 0.5 mL of HMEC medium was then gently dropped onto the top of the solidified gel. Cells were cultured for two weeks and medium was changed every two to three days. Spheroid formation and projection growth were monitored daily under light microscopy. Images were obtained on a Diaphot TMD Nikon Inverted Tissue Culture Microscope using a Spot RT-SE™ camera and SPOT Software Version 4.1 (Diagnostic Instruments Inc).

As described previously [Xu K, et al., 2010, Oncogene 29:6533-42], Matrigel (BD Biosciences) was diluted in 1:1 ratio with ice-cold HMEC medium, and 50 μl of the mixture was placed mid-well in a 24-well plate. After incubating for 5 min in 37° C., an additional 250 μl of Matrigel:medium mixture was added into the well and incubated for another 30 min. A 1:1 mixture of HMEC and RMF cells (0.5×10⁵ cells each) in 0.5 ml of HMEC medium was gently dropped onto the top of the solidified gel. Cells were cultured for two weeks and medium was changed every two or three days. Spheroid formation and projection growth were monitored daily under light microscopy. Images were obtained on a Diaphot Software Version 4.1 (Diagnostic Instruments Inc).

### Example 4

### Organotypic Culture

Three-dimensional human skin equivalents (HSEs) were established as previously described [Andriani F, et al., 2004, Int J Cancer 108:348-57]. Briefly, early passage human foreskin fibroblasts (HFF) were added to neutralized Type I collagen (Organogenesis) mixture to 3.0×10⁴ cells/ml final concentration. Three milliliters of this mixture were added to each 35 mm well insert of a 6-well plate and incubated for 7 days in media containing DMEM and 10% fetal calf serum, until the collagen matrix exhibited no further shrinkage. An amount of 6×10⁵ keratinocytes were then put on top of the contracted collagen gel. Cultures were maintained submerged in low calcium epidermal growth media for 2 days, followed by 2 days in normal calcium medium. Cells were then fed with cornification medium only from the bottom of the well to raise the air-liquid interface. Cornification medium was replaced on days 3 and 5, and tissues were harvested on day 7. Polycarbonate membranes at the bottom of the insert were cut into portions for fixation with 10% formalin overnight and embedded in paraffin. Tissue blocks were sectioned into 8 μm thin sections, mounted, and stained with hematoxylin and eosin (H&E). Images were obtained on a Nikon Eclipse 80i microscope.

### Example 5

### Murine Model of Human Breast Cancer

Eight-week old female non-obese diabetic severe combined immunodeficient (NOD/SCID) mice were purchased from Jackson Laboratory. 2.5×10⁵ SUM1315-GFP/luc cells, with or without co-mixed 7.5×10⁵ RMF cells, were resuspended in Matrigel (BD Biosciences), and injected into 4^{th} inguinal mammary glands in a 350 volume. Animals were supplemented with antibiotics (Septra) in the drinking water for 10 days after surgery. Tumor growth was monitored weekly by manual measurement using electronic digital caliper (Control Company, TX).

### Example 6

### Tiam1 IHC Protocol

Immunohistochemical detection of Tiam1 expression in formalin-fixed paraffin-embedded tissue specimens was performed using the BenchMark XT automated slide preparation system (Ventana edical Systems, Tucson Ariz.). The specific IHC protocol included the following steps: deparaffinization, cell conditioning with conditioner #1 for 30 minutes, manual application of Tiam1 antibody at 1:200 dilution for 32 minutes, amplification, ultraWash, and counterstain with one drop of Hematoxylin for 8 minutes followed by one drop of Bluing Reagent for 4 minutes. The Tiam1 antibody used was rabbit polyclonal IgG from Santa Cruz Biotechnology, Santa Cruz Calif. (catalog #sc-872).

### Example 7

### Tiam1 Deletion in Fibroblasts Affects Epithelial Cell Outgrowth in Mammary Spheroid Co-Culture

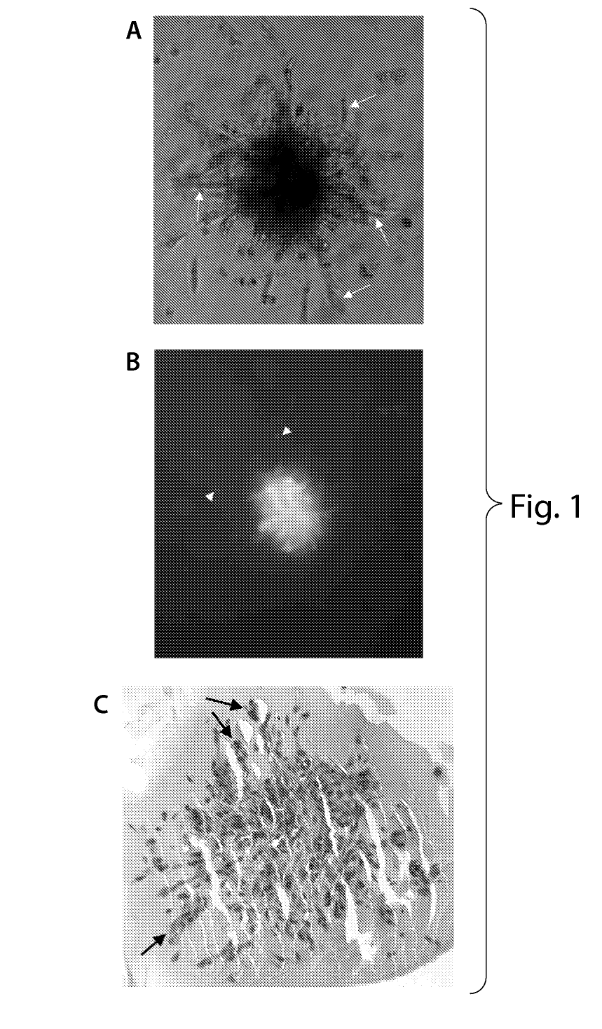

To assess the role of Tiam1 in mammary stromal cells, a three-dimensional in vitro model was utilized allowing co-culture of human mammary-derived fibroblasts with human mammary-derived epithelial cells in an extracellular matrix [Kim J B, 2005, Semin Cancer Biol 15:365-377]. Human mammary epithelial cells (HMECs) and human reduction mammary fibroblasts (RMFs) were used, both derived after immortalization through retroviral delivery of human telomerase (hTERT) [Kuperwasser C, et al., 2004, Proc Natl Acad Sci USA 101:4966-71; Kuperwasser C, et al., 2005, Cancer Res 65:6130-38]. Cells were mixed in a 1:1 ratio and cultured in a Matrigel plug, and were observed to assemble into 3D spheroid structures with fibroblasts clustering in the interior core of the sphere and epithelial cells coating the outside. These examples were initially performed with non-fluorescing HMECs and RMFs expressing GFP. FIG. 1 panel A demonstrates the appearance of one of these spheroids under light microscopy, with green fluorescence of the same field shown in FIG. 1 panel B. The fibroblasts were visualized within the interior core of the spheroid, with non-fluorescing HMECs arrayed around the fibroblast core and assembling into outgrowths projecting into the surrounding matrix (arrows, FIG. 1 panel A). HMECs engineered to express the red fluorescing mCherry protein were used to further demonstrate the location of the HMECs around the periphery and in the projecting outgrowths The projecting outgrowths were multi-cellular, as seen on hematoxylin and eosin staining of fixed, paraffin-embedded spheroids (arrows, FIG. 1 panel C). These spheroids do not progress to hollowed-out gland-like structures such as those described in one-cell-type models of mammary morphogenesis [Debnath J, et al., 2003, J Cell Biol 163:315-326]. However, because the 3D spheroid is composed of close juxtaposition of epithelial cells with stromal fibroblasts, this model permits observation of factors that increase the ability of the epithelial cells to grow out into, i.e., invade the surrounding extracellular matrix.

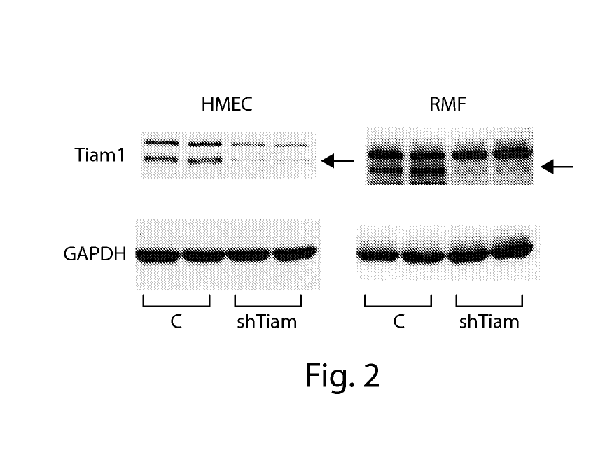

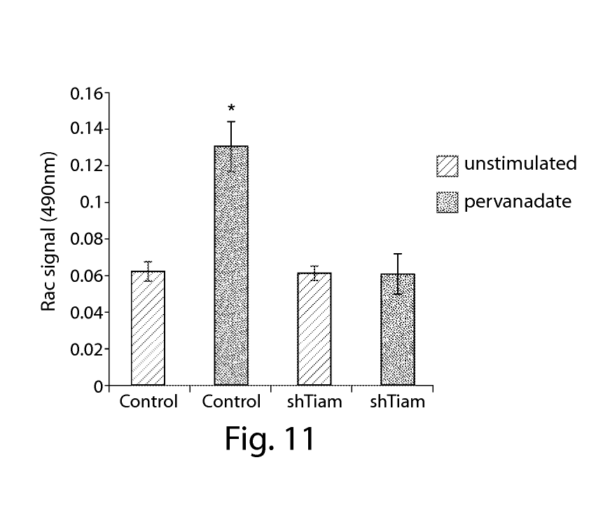

To test whether modulation of Tiam1 levels in breast stromal fibroblasts affects invasiveness in adjacent breast epithelial cells, retroviral delivery of hairpin RNA was used to engineer stable suppression of Tiam1 in either the HMEC line or the RMF line. Tiam1 levels were verified by immunoblotting (FIG. 2, top panels), with equal protein loading verified by immunoblotting for GAPDH (bottom panels). In cells expressing the short hairpin targeting Tiam1 (shTiam), Tiam1 levels were decreased by approximately 75% compared with control cells (C) expressing empty vector (or parental cells, not shown). This sequence was previously used to suppress Tiam1 levels in 293T and NIH3T3 cells (using siRNA oligomers or retroviral hairpin respectively) (Connolly et al., 2005). NIH3T3 fibroblasts with suppressed Tiam1 levels by this method exhibit significantly less Rae activation in response to specific stimuli compared with control fibroblasts [Rajagopal S, et al., 2010, J Biological Chemistry 285:18060-71 incorporated herein by reference in its entirety]. Similarly, RMFs with suppressed Tiam1 expression also showed significantly less Rac activation in response to pervanadate stimulation compared with control fibroblasts (FIG. 11).

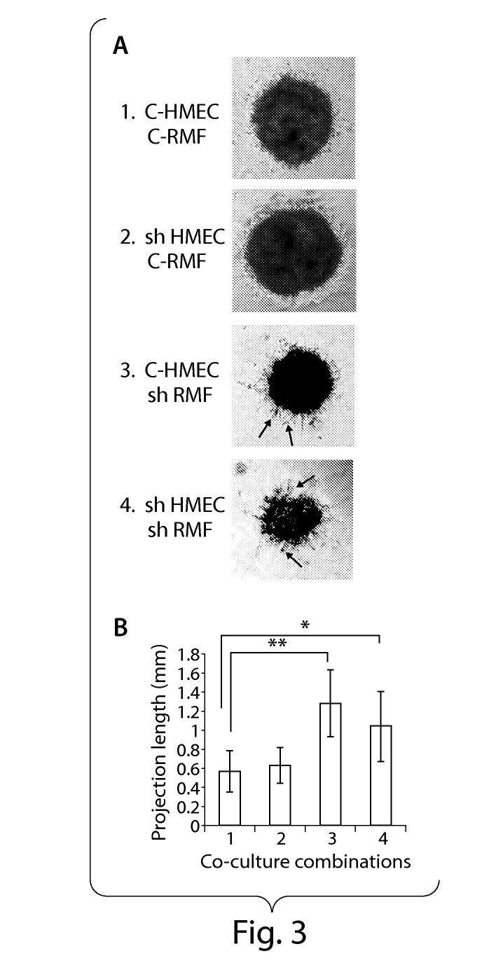

Matrigel co-cultures were established using four possible combinations of control and shTiam1 expression in the HMEC and GFP-RMF lines (FIG. 3 panel A). After 10 days in culture, length of cellular projections into Matrigel was measured under light microscopy (FIG. 3 panel B). Differential Tiam1 expression in HMECs did not affect number or length of projections into Matrigel in the presence of control RMFs (compare combinations 1 and 2). However, Tiam1 silencing in RMFs led to significant enhancement of these epithelial cell outgrowths, regardless of the Tiam1 levels in the co-cultured HMECs (compare combinations 1 and 3). Thus Tiam1 silencing in the fibroblasts, rather than in the epithelial cells, was observed to influence the invasion of the HMECs into the matrix.

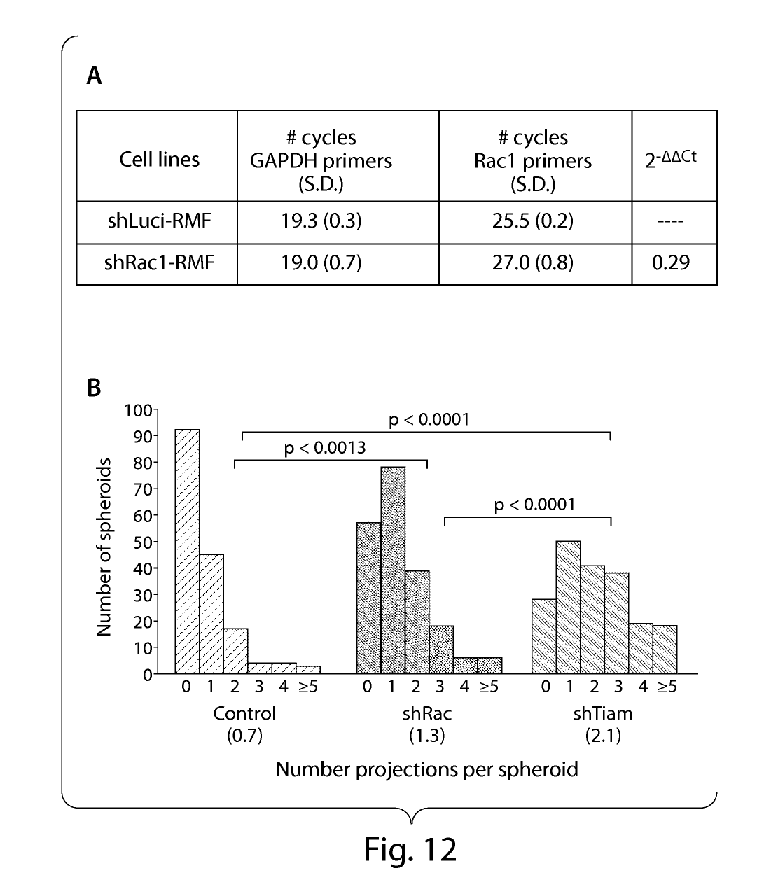

Whether suppression of Rac expression in fibroblasts led to a similar phenotype was tested. Spheroid co-cultures were established with HMECs and either control RMFs or RMFs with stable decrease in Rac1 levels (FIG. 12 panel A). Spheroids from co-culture of HMECs with shRac-RMFs exhibited a somewhat blunted phenotype compared with co-culture of HMECs with shTiam-RMFs developing over the same time frame, exhibiting small projections in somewhat increased numbers compared to control but to a lesser degree than those in shTiam1-RMF co-cultures (FIG. 12 panel B). While Tiam1 deficiency led to decreased Rac activation in cell lysates, Rac deficiency only partially recapitulated Tiam1 deficiency in this three-dimensional assay.

### Example 8

### Tiam1 Depletion in Dermal Fibroblasts Affects Keratinocyte Invasion in a 3D Model of Human Skin

Results above using Tiam1-deficient fibroblasts were extended to engineered tissue to test conditions with more physiologic tissue architecture than spheroid co-culture in Matrigel. The organotypic model of human skin is a well-established tissue model of squamous cell carcinoma progression (Andriani et al., 2004; Garlick, 2007; Segal et al., 2008). In this model, human skin equivalents are fabricated by growing a fully-stratified “epidermis” layered over a stromal “dermis” at an air-liquid interface. In this example the stromal “dermis” is composed of collagen mixed with fibroblasts derived from human foreskin fibroblasts (HFFs). The epithelial “epidermis” is composed of a spontaneously-immortalized human keratinocyte cell line that expresses an activated Ras oncogene (HaCaT-ras-II-4) and forms dysplastic, premalignant epithelium under appropriate culture conditions (Boukamp et al., 1990; Fusenig & Boukamp, 1998).

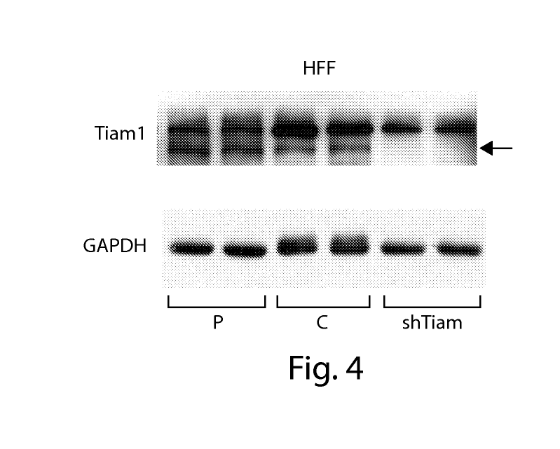

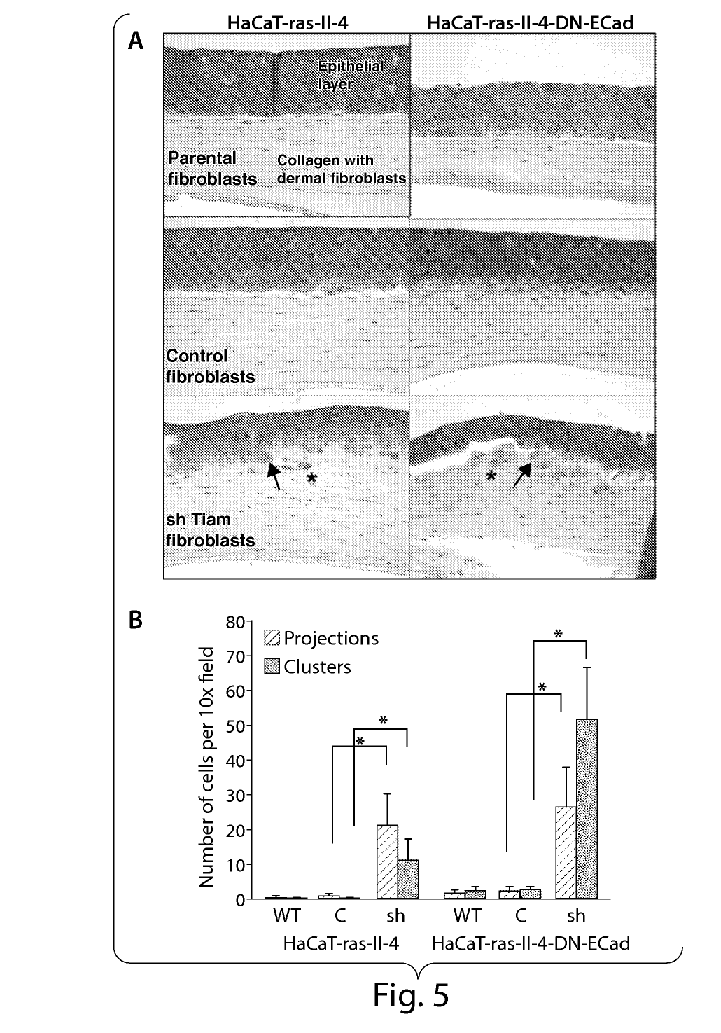

To test the effect of Tiam1 signaling in dermal fibroblasts on HaCaT-ras-II-4 cell invasiveness, HFFs were derived with stable Tiam1 silencing using the same retroviral plasmid hairpin approach as described herein with the HMECs and RMFs. Tiam1 expression was verified by immunoblot and was decreased by 80% compared with parental cells (P) or control vector-containing cells (C) (FIG. 4). Human skin equivalents were established with either parental HFFs, HFFs with control retroviral vector or sh-HFFs in the collagen dermis. In this example two different human keratinocyte cell lines were used. In the first model the keratinocyte layer was established with the HaCaT-ras-II-4 line, originally derived from spontaneously immortalized keratinocytes transformed with Ras, which is not invasive in organotypic culture and displays an intact basement membrane without evidence of cell invasion under control conditions (FIG. 5 panel A, left panels; Boukamp et al., 1990). In the second model the keratinocyte layer was established with HaCaT-ras-II-4-DN-ECad, a more aggressive subline expressing dominant negative E-Cadherin (FIG. 5 panel A, right panels; Margulis et al., 2005).

No difference was observed in invasiveness of either keratinocyte line established over dermal layers containing either parental HFF or HFF transduced with control retrovirus (FIG. 5 panel A, top and middle panels respectively). In contrast, both keratinocyte lines exhibited significantly increased invasiveness into the underlying collagen dermis containing Tiam1-suppressed HFFs (FIG. 5 panel A, bottom panels). In both models, invasion was observed either as projections of groups of cells disrupting the smooth basement membrane and extending out into the collagen layer (projections) or as single cells or small clusters of isolated cells in the collagen layer (clusters). Staining for β-galactosidase, expressed by both keratinocyte lines, confirmed the identity and epithelial nature of the invading cells. Numbers of invading cells were quantified under light microscopy (FIG. 2 panel B). Similar to results herein in the Matrigel co-culture model, suppression of Tiam1 in dermal fibroblasts enhanced epithelial invasiveness, inducing a transition from a premalignant, dysplastic state to a condition showing incipient invasion in this three-dimensional model of human skin.

### Example 9

### Tiam1 Depletion in Breast Stromal Fibroblasts Affects Tumor Invasion in a Mouse Model of Human Breast Cancer

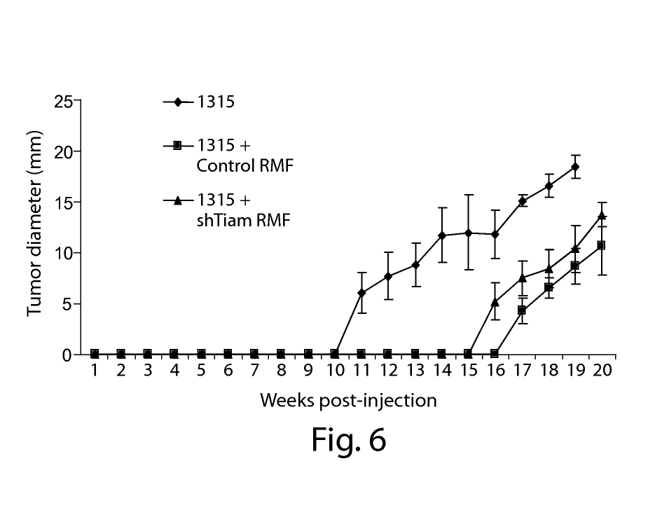

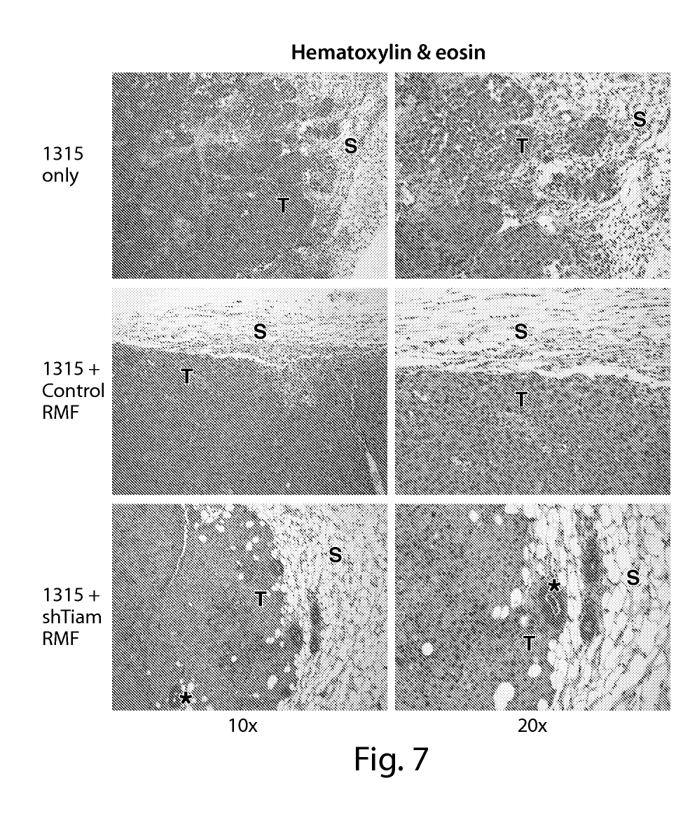

The role of Tiam1 in stromal cells in a mouse model of human breast cancer was next examined [Kuperwasser C, et al., 2004, Proc Natl Acad Sci USA 101:4966-71]. The human breast cancer cell line, SUM1315-GFP/luc, upon injection into mammary fat pads of NOD-SCID mice, yields mammary tumors within a defined time period (8-12 weeks) in 90% of mice (FIG. 6), and spontaneously metastasizes to other organs [Kuperwasser C, et al., 2005, Cancer Res 65:6130-38]. Data here shows that orthotopic tumors from SUM1315-GFP/luc breast cancer cells resulted in lung metastases in approximately 50% of the mice (Table 1). Further, 75% of orthotopic tumors demonstrated areas of invasive growth into surrounding stroma (FIG. 7, top panels). In addition, injection of SUM1315-GFP/luc cells co-mixed with normal mammary fibroblasts inhibited tumor formation during this time frame [Kuperwasser C, et al., 2004, Proc Natl Acad Sci USA 101:4966-71; Willhauck M J, et al., 2007, Carcinogenesis 28:595-610].

TABLE 1
Tumor growth and metastasis in mice bearing human breast cancer
xenografts with and without co-mixtures of mammary fibroblasts.

| | | | #mice with |
|---|---|---|---|
| | # mice with | | detectable lung |
| | tumors/ | # weeks until | mets/ |
| | total mice | measurable tumor | #evaluable |
| | (#tumors) | (s.d) | lungs |
| | (p-value) | (p-value) | (p-value) |
| SUM1315 | 10/10 | 10.8 (1.4) | 4/8 |
| | (16) | (—) | (—) |
| | (—) | | |
| SUM1315 + | 8/10 | 17.1 (2.0) | 0/7 |
| Control RMF | (8) | (p = 0.000006 | (p = 0.03) |
| | (p = 0.66) | | |
| SUM1315 + | 8/10 | 15.8 (2.9) | 5/10 |
| shTiam RMF | (12) | (p = 0.0006) | (p = 1.0) |
| | (p = 0.66) | | |

The effect of Tiam1 suppression in fibroblasts on tumor growth, invasiveness, and metastasis was tested in this model, with the control RMF and shTiam-RMF cells used earlier in the spheroid co-culture model. We found that co-mixture with either fibroblast line decreased orthotopic tumor formation by 25-50% in terms of numbers of mice developing detectable tumors and total number of tumors formed, compared with injection of tumor cells alone (Table 1). Tumor development was also delayed to a similar extent after co-mixture with either fibroblast line, with time to first measurable tumor being significantly delayed in these mice (Table 1 and FIG. 6). Thus tumorigenesis was decreased by the presence of co-injected fibroblasts, and this effect was independent of fibroblast Tiam1 expression.

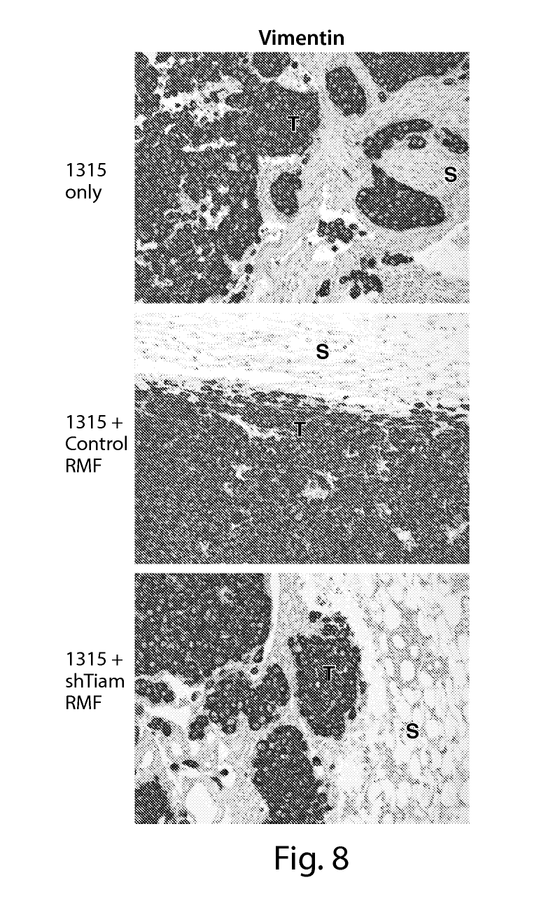

However, the histology of the tumors was notably different at the interface between tumor and surrounding stroma depending on Tiam1 status in the associated fibroblasts (FIG. 7). All tumors developing in mice implanted with Sum 1315-GFP/luc cells co-mixed with control RMF demonstrated a “pushing” smooth border between tumor cells and adjacent stroma, with less evidence of stromal invasion by tumor cells (FIG. 7, middle panels). In mice implanted with SUM1315-GFP/luc cells co-mixed with shTiam-RMF, 75% of tumors exhibited a more infiltrative, invasive tumor-stromal border, with tumor cells extending out into the surrounding stroma and around murine mammary structures (FIG. 7, bottom panels), similar to the pattern seen with implantation of Sum 1315-GFP/luc cells alone. In this model, the tumor cells express vimentin, and immunohistochemical staining for human-specific vimentin readily demonstrated the presence of tumor cells invading into surrounding stroma after injection of SUM1315-GFP/luc cells alone (FIG. 8, top panel) or in association with Tiam1-suppressed fibroblasts (bottom panel), as opposed to with control fibroblasts (middle panel).

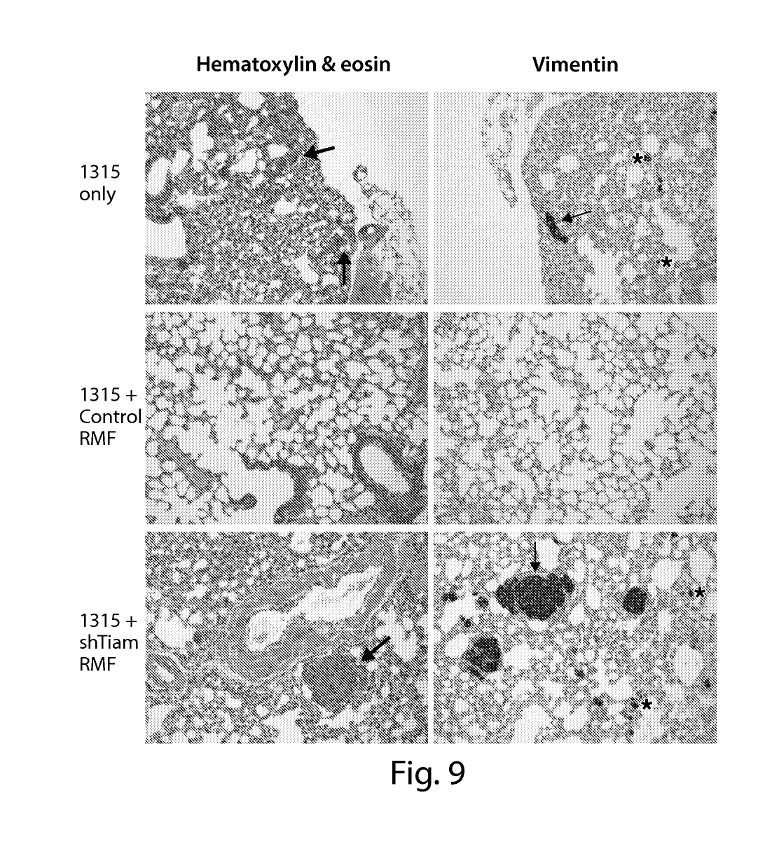

Whether the degree of tumor invasion observed on histopathologic examination of the tumor correlated with metastatic behavior was determined. Half the mice implanted with SUM1315-GFP/luc alone were observed to have lung metastases, detectable either as tumor nodules visible on routine histopathology or as isolated tumor cells detected by vimentin immunostaining (Table 1 and FIG. 9, top panels). In contrast, no mice with establishment of orthotopic tumors from SUM1315-GFP/luc breast cancer cells co-mixed with control fibroblasts exhibited detectable tumor cells within their lungs (middle panels). Further, 50% of the mice receiving shTiam RMF along with SUM1315-GFP/luc breast cancer cells had detectable tumor cells in their lungs by routine histology or vimentin immunostaining, similar to mice receiving the tumor cells alone (bottom panels). Thus, while tumorigenesis was not affected, Tiam1 suppression in breast stromal fibroblasts significantly increased invasiveness and metastatic potential of the breast tumors in this model.

### Example 10

### Human Correlation: Fibroblast Tiam1 Expression Varies Inversely with Invasiveness in Human Breast Cancer

A highly significant difference in Tiam1 expression in fibroblasts adjacent to tumors was found in human breast cancer samples. A significant number of cases of high-grade Ductal Carcinoma In Situ (DCIS) (non-invasive cancer) have been examined for Tiam1 expression. In in more than 85% of DCIS samples, Tiam1 was found to be expressed in the fibroblasts located adjacent to the tumor. In an equal number of invasive cancer cases similarly examined for Tiam1 expression, none of the invasive cancer samples were found to have Tiam1 expression in fibroblasts located adjacent to the tumor.

The role of Tiam1 in stromal fibroblasts of the tumor microenvironment on epithelial cell invasiveness was investigated. The tissue microenvironment includes a complex network of intercellular interactions that are mediated by physical attachment, as in direct cell-cell or cell-extracellular matrix (ECM) interactions, and by biochemical signals, mediated by soluble molecules. Monolayer, 2D culture systems do not generate spatially-organized, 3D structures that occur in vivo. Multiple cell functions are affected by dimensional context, including cell shape and polarity, growth, morphogenesis, differentiation, and gene expression. Factors affecting the outcome in different models include use of cells in single suspension vs. aggregates, nutrient restrictions, composition and stiffness of extracellular matrix, and cell polarity.

Examples herein shed light on the role of Tiam1 in mammary fibroblasts; however, different tissue models with a range of technical complexity and biologically meaningful tissue context were used in order to enhance the significance of the findings and allow a generalization to stromal fibroblast-associated epithelial cell interactions. As demonstrated herein, Tiam1 expression in stromal fibroblasts affects the invasive behavior of associated epithelial cells. In spheroid co-culture of HMECs and RMFs, epithelial cells exhibited increased invasiveness into the surrounding extracellular matrix when Tiam1 was suppressed in the fibroblasts. Similarly, in organotypic cultures of engineered human skin fabricated with epidermis from premalignant keratinocytes and dermis comprised of collagen mixed with skin fibroblasts, two different keratinocyte lines exhibited significantly more invasion into the dermis when Tiam1 levels were suppressed in the dermal fibroblasts.

Extending this study into a more complex whole animal system, a murine model of human breast cancer, yielded more complex findings. Co-injection of any mammary fibroblasts retarded tumorigenesis, invasiveness, and lung metastasis, compared with establishment of tumors in the absence of fibroblasts. While the effects on tumorigenesis were independent of stromal Tiam1 levels, breast tumor invasion and metastasis were significantly increased in the presence of fibroblasts with suppressed levels of Tiam1. Tiam1 suppression reversed the stromal inhibition of tumor invasion and metastasis and did not affect the stromal inhibition of tumorigenesis itself. Thus, fibroblasts of the microenvironment have unexpected and paradoxical effects on associated tumors that are governed by more than one set of signaling pathways. A similar complexity is seen in the paradigm in tumor cells that multiple distinct pathways are involved in acquisition of the characteristics needed for malignant transformation [Hanahan D, et al., 2000, Cell 100:57-70].

Fibroblasts are the predominant cell type in stromal connective tissue, contributing to deposition and maintenance of basement membrane and paracrine growth factors. In addition, a strategy for studying the role of specific signaling molecules and pathways in the tumor microenvironment in the evolution of cancers is provided herein. As shown herein, the finding that signaling in breast stromal fibroblasts can affect not only local invasiveness but also organ metastasis means that stromal effects on associated tumor cells persist beyond the time of direct tumor-stroma contact. Understanding the details underlying this could lead to new therapeutic strategies for treating and preventing breast cancer metastasis

### Example 11

### Generation of Cell Lines

All oligomers used for engineering stable lines were synthesized in the Tufts DNA Core Facility. HMECs with red fluorescence through expression of mCherry, RMFs with green fluorescence through expression of eGFP, the Tiam-deficient shTiam-RMF line and the retroviral hairpin control C-RMF line have been described previously [Xu K, et al., 2010, Oncogene 29:6533-42].

The cDNA for full length Tiam1 was synthesized in two segments corresponding to bp 1-1948 and bp 1894-4773 using PCR amplification of a full-length Tiam1 cDNA template, using the following primers:

5′ segment: Sense
(SEQ ID NO: 4)
5′ CGGGATCCATGGGAAACGCAGAAAGTCAA
anti-sense
(SEQ ID NO: 5)
5′CCACTTTCGTTGTCGACT
3′ segment: Sense
(SEQ ID NO: 6)
5′GAGCTGCCAAACCCCAAA
anti-sense
(SEQ ID NO: 7)
5′ATAGTCGACGATCTCAGTGTTCAGTTTCCTC

The 5′ segment was first ligated into pBabepuro using BamH1 and Sal1 restriction enzyme digestion, taking advantage of an internal Sal1 site. The 3′ segment was then ligated into the resulting product downstream of the first segment at the Sal1 site. Correct orientation was validated by DNA mapping and sequencing. RMF cells were then transfected with pBabepuro-Tiam1 or control pBabepuro plasmid and stable integrants were selected by culturing in complete medium containing 0.5 μg/ml puromycin. Expression of Tiam1 in pBabepuro-Tiam1 colonies was validated by immunoblot.

The cDNA for full-length Tiam1 was cloned into the pBI-G Tet vector (Clontech) using a similar strategy as above, with the exception that the sense primer of the 5′ segment incorporated a Not1 site, with the following sequence: 5′ segment: Sense 5′ATAAGAAGCGGCCGCATGGGAAACGCAGAAAGTCAA (SEQ ID NO: 8); and the first ligation used Not1/Sal1 digestion. pBI-G-Tiam1 or pBI-G control vector were transfected into MEF/3T3 cells carrying the tetracycline-controlled transactivator tTA regulatory protein (Tet-Off; Clontech), and stable transformants were selected in hygromycin (Clontech).

RMF lines with stable expression of short hairpin RNAs targeting OPN or luciferase control were generated using the pENTR/U6-pLentib/BLOCK-iT lentiviral RNAi expression vector system (Invitrogen), using the following hairpin oligomers:

OPN Sense:
(SEQ ID NO: 9)
5′CACCCTTTACAACAAATACCCAGATTTCAAGAGAATCTGGGTATT
TGTTGTAAAG
OPN Anti-sense:
(SEQ ID NO: 10)
5′AAAACTTTACAACAAATACCCAGATTCTCTTGAAATCTGGGTATT
TGTTGTAAAG

For production of lentivirus, each of pLenti6/BLOCK-iT-OPN plasmid DNA, and pLenti6/BLOCK-iT-luci plasmid DNA, were transfected into 293FT cells along with pLP1, pLP2, and pVSV-G DNAs using Lipofectamine (Invitrogen) and virus-containing supernatant was harvested according to manufacturer's instructions. Recipient RMF cells were infected with filtered viral supernatants in the presence of 6 μg/ml polybrene and stable transformants were selected in blasticidin (Invitrogen). Silencing of OPN in the shOPN-RMF line was verified by qRT-PCR.

An RMF line with stable silencing of both Tiam1 and OPN was generated by transducing the shTiam-RMF line with lentiviral particles harvested from 293FT cells transfected with the pLenti6/BLOCK-iT-OPN plasmid DNA as described above. A double viral control line with both retroviral and lentiviral mediated antibiotic resistance was generated by transducing the C-RMF line with lentiviral particles harvested from cells transfected with the pLenti6/BLOCK-iT-luci DNA as described above, and selected in G418 and blasticidin. Tiam1 silencing was verified by immunoblot; OPN silencing was verified by qRT-PCR.

### Example 12

### Antibodies and Immunoblotting

Antibodies to Tiam1, OPN, and GAPDH (Santa Cruz) were used according to manufacturers' instructions. Preparation of cell lysates, protein gel electrophoresis and transfer, and secondary antibodies have been previously described [Buchsbaum, R, et al. 1996, Mol Cell Biol 16:4888-96]. After washing in PBS, cells were lysed in SP buffer containing 50 mM Tris, pH 8.0, 120 mM NaCl, 1 mM EDTA, 0.5% NP-40 along with protease inhibitors (10 μg/ml of aprotinin, 20 uM leupeptin, 3 mM phenylmethylsulfonyl fluoride (PMSF) and phosphatase inhibitors (50 μM sodium fluoride and 100 μM sodium orthovanadate (NaV)). Protein bands were visualized by chemiluminescence using Western Lightning™ Plus-ECL Kit (PerkinElmer).

### Example 13

### Real Time PCR

Total RNA and first strand cDNA synthesis were carried out using the TRIzol™ and SuperScript™ (Invitrogen) protocols respectively per manufacturer instructions. PCR was performed in triplicate reactions in 25-ul volumes containing cDNA, SYBR Green PCR Mastermix (Applied Biosystems). The primer sets used for the quantitative PCR analysis are listed below:

Tiam1 (human) Sense
(SEQ ID NO: 11)
5′ AAGACGTACTCAGGCCATGTCC
Antisense
(SEQ ID NO: 12)
5′ GACCCAAATGTCGCAGTCAG
OPN (human) Sense
(SEQ ID NO: 13)
5′ GCCATACCAGTTAAACAGGC
Antisense
(SEQ ID NO: 14)
5′ GACCTCAGAAGATGCACTAT
OPN (mouse) Sense
(SEQ ID NO: 15)
5′ CTCCCGGTGAAAGTGACTGA
Antisense
(SEQ ID NO: 16)
5′ GACCTCAGAAGATGAACTCT
GAPDH (Human) Sense
(SEQ ID NO: 17)
5′ CTGCACCACCAACTGCTTAG
Antisense
(SEQ ID NO: 18)
5′ TTCAGCTCAGGGATGACCTT
β actin (mouse): Sense
(SEQ ID NO: 19)
5′ TGGAATCCTGTGGCATCCATGAAAC
Antisense:
(SEQ ID NO: 20)
5′ TAAAACGCAGCTCAGTAACAGTCCG

Real-Time PCR Parameters Used were as Follows:
PCR for amplication of OPN: 95° C. for 10 min; 95° C. for 30 s, 60° C. for 60 s, 72° C. for 60 s for 40 cycles. PCR for amplication of Tiam1: 94° C. for 10 min; 94° C. for 30 s, 58° C. for 40 s, 72° C. for 90 s for 45 cycles. Data analysis was done using an Opticon™ 2 continuous Fluorescence Detector (MJ Research). The 2-ΔΔ-Ct value was calculated following glyceraldehyde 3-phosphate dehydrogenase (GAPDH) or β-actin normalization.

### Example 14

### Flow Cytometry

Two to three weeks after isolation from co-culture, cells were trypsinized into single cell suspension, washed, and resuspended in PBS. Cells were analyzed on a DakoCytomation New Cyan ADP using the Summit 4.3 program, with X-axis set to FITC Log Comp to detect cells containing eGFP and Y-axis set to PE-Texas Red Log Comp to detect cells containing mCherry.

### Example 15

### Transwell Migration Assays

Cultured cells were deprived of serum overnight, trypsinized, and plated at a density of 1×10⁵/ml (2×10⁴ cells/basket) in the upper basket of transwell chambers with a filter pore size of 8 um (Costar). Cells were allowed to migrate for 5 hours at 37° C. toward lower chambers containing either Dulbecco's modified Eagle's medium alone or supplemented with 25% filter-sterilized conditioned medium harvested from NIH3T3 cells. Non-migrated cells were then removed from the upper side of the filter using a cotton swab. Filters were fixed and stained with the Protocol 3® HEMA Stain kit (Fisher). Filters were cut out and mounted on glass slides under coverslips using Resolve microscope immersion oil (Richard Allen Scientific). Migrated cells were counted in nine random fields using a Nikon Eclipse TS100 microscope and 20× objective.

### Example 16

### Seeded Cell Migration Assay

Indicated RMF cells were seeded on the bottom of the lower chamber one day before the migration assay at a density of 2×10⁴ cells/chamber and were returned to the incubator overnight to reach approximately 70% confluence. OPN antibody (Santa Cruz) or rabbit IgG were added as indicated to the lower chamber at a concentration of 1 mg/ml immediately after RMFs were seeded.

### Example 17

### Senescence Induction and SA-β-Gal Staining

To induce senescence, 2.5×10⁵ RMF cells were seeded on 100-mm plates for 48 hours until approximately 80% confluent and then treated with either 800 μmol/L hydrogen peroxide (H₂O₂) for 2 hours or 50 μg/ml bleomycin in culture medium for 24 hours at 37° C. After treatment, cells were rinsed twice with phosphate-buffered saline (PBS) and left to recover in culture medium. For radiation-induced senescence, cells were subjected to 16Gy X-irradiation (233 cGy/min for 6 min 52 sec), and returned to incubator to recover. For each treatment, senescence induction was repeated 3-5 days later to prevent recovery and cell cycle re-entry. Cells were sub-cultured for at least 7 days and senescence induction was confirmed by SA-β-gal staining.

SA-β-Gal staining was conducted as described previously [Dimri, G P, et al., 1995, Proc Natl Acad Sci USA 92:9363-7]. Briefly, cells were washed twice in PBS and fixed 5 minutes in 2% formaldehyde/0.2% glutaraldehyde, washed again, and incubated at 37° C. overnight with fresh senescence-associated β-Gal (SA-β-Gal) stain solution: 1 mg 5-bromo-4-chloro-3-indolyl-β-D-galactoside (X-Gal) per ml (stock=20 mg in dimethylformamide per ml)/40 mM citric acid/sodium phosphate, pH 6.0/5 mM potassium ferrocyanide/5 mM potassium ferricyanide/150 mM NaCl/2 mM MgCl2. Cells with positive staining were observed and counted under a light microscope (Nikon, Eclipse TS100).

### Example 18

### Calpain Activity Assay

Calpain activation was assessed by fluorometric detection of cleavage of calpain substrate using a purchased Calpain Activity Assay Kit (Biovision Research Products) according to manufacturer's instructions. Briefly, equal numbers of cells were lysed in supplied extraction buffer and post-centrifugation supernatants were normalized for protein content in extraction buffer. After addition of supplied reaction buffer and calpain substrate (Ac-LLY-AFC), samples were incubated in the dark for 1 hour at 37° C. Samples were transferred to 96-well plates and fluorescence was detected using a Victor3™V1420 Multilabel Counter and Wallac Victor 3V software (Perkin Elmer) equipped with a 405 nm excitation filter and 535 nm emission filter. Results were corrected for background fluorescence as measured in empty wells. In some samples calpain substrate was omitted (negative control), supplied calpain inhibitor was added, or supplied active calpain was added to extraction buffer (positive control).

### Example 19

### In Vitro Tiam1 Cleavage Assay

HEK293T cells were transiently transfected with full-length Tiam1 cDNA as previously described (Buchsbaum et al., 2002). Forty-eight hours after transfection, cells were washed once with PBS and lysed in SP buffer as described above. Lysates were cleared of unbroken cells and debris by centrifugation at 10,000×g for 10 minutes. Aliquots of cleared lysates were reserved for immunoblots; the remainder were incubated with protein A-Sepharose beads (Pharmacia) and anti-Tiam1 antibody (diluted according to the manufacturer's instructions) or equal amounts of polyclonal rabbit IgG (Santa Cruz) for 2 h at 4° C. with constant agitation. Immunoprecipitates were washed twice with ice-cold SP buffer and once with Reaction Buffer (20 mM Tris-HCl, pH 7.5, 10 mM DTT, 6 mM CaCl₂) in the presence of protease inhibitors (1 mM PMSF and 1.7 μg/ml aprotinin) prior to the cleavage reaction.

Pre-senescent cells were maintained in culture as described above. Senescence was induced with 800 uM H₂O₂ as described above, and confirmed by SA-β gal staining. Pre-senescent and senescent RMFs were washed with cold PBS, pelleted, and resuspended in extraction buffer (10 mM HEPES pH 7.0, 2 mM MgCl₂, 50 mM NaCl, 1 mM DTT) containing inhibitors (17 μg/ml aprotinin, 10 μg/ml leupepsin, 100 μM NaV, 3.3 mM PMSF). Cells were lysed by freeze-thaw cycles in ethanol-liquid nitrogen/37° C. water bath. Extracts were centrifuged at 10,000 g for 15 min at 4° C., and resulting supernatants were used as the cytosolic fraction. Protein concentration was determined by BCA protein assay (Bio-Rad).

Immunoprecipitated proteins were incubated with extracted lysates from pre-senescent or senescent cells in Reaction Buffer for 2 hours at 37° C. with constant agitation. Where indicated, extracted lysates were pre-incubated on ice for 30 minutes with calpain inhibitor (50 μM ALLN) or 1 mM EDTA, or with proteasome inhibitor (5 nM bortezomib) for 37° C. for 24 hours before the cleavage reaction. (Proteasome inhibition under these conditions was verified using the Proteasome-Glo Cell-Based Assay and GloMax®-multi+Detection system [Promega] according to manufacturer's instructions.) The beads were then washed 3 times with Tris-HCl pH 7.5 and the reaction was stopped by addition of 6× Laemmli buffer and heating. Samples were resolved by SDS-PAGE and immunoblotting as described.

### Example 20

### Spheroid Co-Culture in Matrigel

As described previously [Xu K, et al., 2010, Oncogene 29:6533-42], Matrigel (BD Biosciences) was diluted in 1:1 ratio with ice-cold HMEC medium, and 50 ul of the mixture was placed mid-well in a 24-well plate. After incubating for 5 min in 37° C., an additional 250 μl of Matrigel:medium mixture was added into the well and incubated for another 30 min A 1:1 mixture of HMEC and RMF cells (0.5×10⁵ cells each) in 0.5 ml of HMEC medium was gently dropped onto the top of the solidified gel. Cells were cultured for two weeks and medium was changed every two or three days. Spheroid formation and projection growth were monitored daily under light microscopy. Images were obtained on a Diaphot Software Version 4.1 (Diagnostic Instruments Inc).

### Example 21

### Isolation of Cells from Spheroid Co-Culture

Co-cultured spheroids were removed from Matrigel by gentle pipetting using 1000 uL plastic pipet tips with the tip cut off, transferred to sterile 15 ml centrifuge tubes, and centrifuged at 2000 rpm for 5 min. The disrupted Matrigel was gently removed from the top of the tubes and the pelleted cells and spheroids were transferred in HMEC medium to 35 mm wells. Cell/spheroid mixtures were cultured for 7-10 days until cells lost the spheroid structure and became monolayers, and then expanded when reaching 50% confluence.

### Example 22

### Osteopontin mRNA and Protein Expression are Inversely Correlated with Tiam1 Protein Expression in Fibroblasts

To investigate how Tiam1 expression in tumor-associated fibroblasts could affect invasiveness of associated tumor cells, gene expression analysis was performed in two groups of cell lines with altered Tiam1 expression using Affymetrix microarrays: human reduction mammary fibroblasts (RMFs) with stable silencing of Tiam1 (shTiam-RMF) compared with control RMFs (C-RMF), and mouse embryo fibroblasts (MEFs) with inducible Tiam1 expression (+Tiam-MEF) compared with induced control MEFs, respectively. Microarray data were analyzed using Ingenuity Pathway Analysis, and significantly changed genes were compared between the RMF group and the MEF group for inverse patterns of expression. Significantly changed genes in either Tiam1-deficient RMFs or Tiam1-expressing MEFs included several cytokines and extracellular matrix proteins.

Of these, only the osteopontin gene showed corresponding inverse expression in the two cell lines, being consistently up-regulated in Tiam1-deficient fibroblasts and consistently down-regulated in Tiam1-over-expressing fibroblasts. OPN is a secreted glycoprotein and many of its effects are mediated through NFκB signaling [Wai P Y, et al., 2004, J Surg Res 121:228-41]. Pathway analysis also revealed increased expression of multiple NFκB pathway components in Tiam1-deficient fibroblasts. Given the association of increased. OPN expression with tumor progression and metastasis [Wai, P Y, et al., 2008, Cancer Metastasis Rev 27:103-18], OPN was selected as a potential mediator of Tiam1 effects in the tumor microenvironment.

OPN was further analyzed for mRNA levels in shTiam-RMF cells using qRT-PCR. OPN mRNA was observed to be up-regulated in shTiam-RMF compared to C-RMF (FIG. 13 panel A). The amount of secreted OPN was also assessed by immunoblots of conditioned media. OPN protein levels were consistently increased 2-3× in conditioned medium from Tiam1-deficient RMF compared with control RMF (FIG. 13 panel B).

OPN mRNA expression was tested in the inducible +Tiam1-MEF line. After removal of doxycycline from culture medium, Tiam1 protein over-expression was confirmed by immunoblots. Real-time-PCR using OPN-specific primers confirmed that OPN mRNA was significantly decreased in the presence of Tiam1 over-expression compared with doxycycline-deprived MEF-pBIG control cells. To validate that this converse correlation also occurs in human fibroblasts, an RMF cell line was construed with stable high expression of Tiam1 (+Tiam-RMF). These cells exhibited significant decrease in OPN mRNA levels (FIG. 13 panel C) as well as in secreted protein (FIG. 13 panel D), compared with control cells. These results confirm the results of the microarray and indicate that OPN mRNA and protein expression are inversely correlated with Tiam1 protein expression in human fibroblasts.

### Example 23

### RMFs Undergo Stress-Induced Senescence

Senescent fibroblasts can induce an epithelial-mesenchymal transition (EMT) in associated tumor cells and display up-regulated OPN (Krtolica et al., 2001; Pazolli et al., 2009). We have found that Tiam1-deficient fibroblasts induce increased invasion and metastasis in associated tumor cells [Xu K, et al., 2010, Oncogene 29:6533-42]. Stress-induced senescence was tested for ability to lead to down-regulation of Tiam1 in fibroblasts.

Whether RMF cells, which are immortalized by telomerase expression, could undergo stress-induced senescence, was investigated. Several different inducers, including oxidative stress (hydrogen peroxide) or sub-lethal DNA damage (the chemotherapeutic agent bleomycin or radiation) [Aoshiba K, et al., 2003, Eur Respir J 22:436-43, Bavik C, et al, 2006, Cancer Res 66:794-802, Hornsby P J, 2007, J Clin Oncol 25:1852-7, Parrinello S, et al., 2005, J Cell Sci 118:485-96] were tested. Seven days after induction, cells treated with either H₂O₂, bleomycin, or radiation had taken on a morphologic appearance characteristic of senescence, appearing flattened and enlarged, and did not undergo either proliferation or apoptosis for at least 2 weeks [Krtolica A, et al., 2002, Int J Biochem Cell Biol 34:1401-14]. Almost all the cells were observed to have exhibited senescence-associated β-galactosidase (SA β-gal) staining, which is known to indicate effective induction of senescence by each stress [Dimri G P, et al., 1995, Proc Natl Acad Sci USA 92:9363-7]. Tiam1 expression did not affect induction of senescence.

### Example 24

### Stress-Induced Senescence Leads to Inverse Changes in OPN and Tiam1 in Fibroblasts

OPN expression was observed to have increased in senescent RMF cells, similar to previous reports in senescent foreskin fibroblasts [Pazolli E et al., 2009, Cancer Res 69:1230-9]. OPN mRNA levels were significantly increased in cells induced to senesce by either oxidative or chemical stress (FIG. 2A). Furthermore increases in secreted OPN were also seen in conditioned medium harvested from cells after induction of senescence by either stress (FIG. 14 panel B).

Tiam1 expression was assessed in RMFs undergoing stress-induced senescence. In contrast to the results with OPN, no significant difference in Tiam1 mRNA was observed between pre-senescent and senescent cells (FIG. 14 panel C). A notable decrease was observed in Tiam1 protein in cells that had undergone either oxidative or chemical stress-induced senescence (FIG. 14 panel D). The effect of senescence induction on cells with increased baseline Tiam1 expression using the +Tiam-RMF line was tested. Tiam1 mRNA was significantly higher in +Tiam1-RMF cells than in control RMF cells, and did not change with senescence induction (FIG. 14 panel E). However, Tiam1 protein levels in these cells also decreased significantly upon senescence induction (FIG. 14 panel F). These results indicate that stress-induced senescence leads to both increases in OPN and decreases in Tiam1 protein. Oxidative stress was used to induce senescence for additional examples herein.

### Example 25

### Tiam1 Protein is Likely Degraded by Calpain Protease During Stress-Induced Senescence in Cells

The findings on Tiam1 mRNA and protein expression suggest post-transcriptional regulation of Tiam1 in cells undergoing senescence. Several signaling pathways and proteases have been reported to be involved in the degradation of Tiam1 protein, in particular activation of calcium-dependent calpain proteases [Qi H, et al., 2001, Cell Growth Differ 12:603-11; Woodcock S A, et al., 2009, Mol Cell 33:639-53]. Induction of senescence in various cell types triggers a DNA damage response that then triggers activation of calpain proteases [Demarchi F, et al., 2010, Cell Cycle 9:755-60].

To explore whether calpains might be involved in Tiam1 down-regulation in senescent cells, whether calpain activation was increased in cells undergoing induced senescence was tested. While pre-senescent cells exhibited some calpain activity at baseline, this was increased over 2-fold in cells undergoing stress-induced senescence. We then asked whether inhibition of calpain proteases would block the decrease in Tiam1 seen in senescent cells was tested. Treatment of cultured cells with the calpain inhibitor ALLN during induced senescence led to toxic cell death at all doses tested. Therefore an in vitro Tiam1 cleavage assay based on similar in vitro protease assays reported previously [Juo P, et al., 1998, Curr Biol 8:1001-8; Li H, et al., 1997, Journal of Biological Chemistry 272:21010-7; Qi H, et al., 2001, Cell Growth Differ 12:603-11; Woodcock S A, et al., 2009, Mol Cell 33:639-53] was performed. Tiam1 immunoprecipitates from cells with exogenous Tiam1 expression were incubated with lysates from either pre-senescent or senescent cells, and levels of immunoprecipitated Tiam1 remaining post-incubation were assessed by immunoblot (FIG. 3A). In cells with high levels of exogenous Tiam1, the protein often migrates on protein gels as a double band attributed to partial protein degradation. Incubation of immunoprecipitated Tiam1 with any cellular lysates led to some degradation compared with non-incubated Tiam1 precipitate (compare ratio of upper to lower bands in lane 1 with lanes 3-8). However, the overall amount of precipitated Tiam was notably decreased by incubation with lysate from senescent cells (compare lanes 3 and 6). Pre-incubation of cell lysates with either the calpain inhibitor ALLN or the calcium-chelator EDTA significantly inhibited degradation of the immobilized Tiam1 induced by senescent lysates (lanes 7 and 8 respectively). In contrast, pre-incubation of cell lysates with the proteasome inhibitor bortezomib did not prevent degradation of immobilized Tiam1 by senescent lysates (FIG. 15 panel B, compare lanes 11-12 with lanes 13-14). These results suggest that Tiam1 down-regulation in cells undergoing senescence is likely due at least in part to calpain-mediated protein degradation.

### Example 26

### Tiam1 is Inversely Correlated with OPN Expression

As OPN expression is increased and Tiam1 expression is decreased in senescent cells, then Tiam1 levels may influence regulation of OPN expression. The effect of Tiam1 over-expression on OPN levels in cells was assessed. As in FIG. 14, induction of senescence in control cells led to an increase in OPN mRNA (FIG. 16 panel A, compare bars 1 and 2). In +Tiam-RMF cells, baseline levels of OPN were suppressed compared to control cells (compare bars 1 and 3). Upon induction of senescence, OPN levels were observed to increase, and to a much lesser extent than in cells with wild-type Tiam1 expression (compare bars 2 and 4). Further, variation in OPN levels did not affect Tiam1 expression. In cells with stable silencing of OPN (shOPN-RMF), Tiam1 protein levels were unaffected at baseline (FIG. 16 panel. B, compare bars 1 and 3). In these cells, Tiam1 levels also decreased to a similar extent as in control cells upon senescence induction (FIG. 16 panel B, compare bars 2 and 4). Taken together with the results in FIG. 13, these results show that Tiam1 expression inversely regulates expression of OPN.

### Example 27

### Senescent Fibroblasts Promote Invasion and Migration of Associated Epithelial Cells

As data herein show that senescent fibroblasts have decreased Tiam1 and increased OPN similar to Tiam1-deficient fibroblasts, whether senescent fibroblasts could also promote epithelial cell invasion in three-dimensional culture was tested. To differentiate between cell lines in mixed cell spheroid co-cultures, immortalized human mammary epithelial cells (HMECs) engineered with red fluorescence through stable expression of mCherry and RMFs with green fluorescence through stable expression of GFP were used. Data herein show that in mixed cell spheroid co-cultures with HMECs and RMFs, the fibroblasts cluster in the interior of the spheroid while the epithelial cells are located around the periphery of the spheroid. Under conditions promoting increased invasiveness, HMECs form increased numbers of multi-cellular projections invading into the matrix [Xu K, et al., 2010, Oncogene 29:6533-42].

In preliminary examples HMECs were observed to exhibit increased invasiveness into the surrounding matrix when cultured with RMFs rendered senescent by exposure to either hydrogen peroxide or bleomycin, similar to the invasiveness induced upon co-culture with Tiam1-deficient fibroblasts. A protocol was optimized for isolating HMEC cells out of spheroid co-culture through pipetting and serial passage. This protocol yields HMEC populations with >98% purity within two weeks after extraction out of co-culture, based on flow cytometry. It was observed that HMECs isolated after co-culture with Tiam1-deficient RMFs (termed post-co-culture HMECs) exhibit increased motility in transwell migration assays. HMECs isolated after co-culture with senescent fibroblasts also exhibit increased motility to a similar extent. This increased motility persisted for >12 weeks after isolation, indicating long-term effects of co-cultured fibroblasts on associated epithelial cells.

### Example 28

### Up-Regulation of Tiam1 in Senescent RMF Cells Inhibits the Invasion and Migration of Associated Epithelial Cells

As Tiam1 expression is decreased in cells undergoing senescence, whether up-regulation of Tiam1 could block the increased epithelial cell invasiveness induced by senescent fibroblasts was examined.

In co-cultures of HMECs with senescent RMFs, spheroids were observed to display increased HMEC invasion into Matrigel as assessed by numbers of HMEC projections per spheroid (FIG. 17, panels A-H, compare panels C and G; quantified in Q, compare bars 1 and 2). Epithelial cells isolated from co-cultures displayed significantly increased migration when isolated from co-cultures with senescent fibroblasts compared with non-senescent fibroblasts (panel R, compare bars 1 and 2). In co-cultures of HMECs with senescent Tiam-overexpressing +Tiam-RMF cells, there was some blunting in numbers of projections per spheroid compared with senescent RMFs, with increased numbers of spheroids with 0-1 projection and decreased number of spheroids with ≧5 projections (panels I-P and Q, compare bars 2 and 4). In addition, while migration of HMECs isolated from co-cultures with +Tiam-RMF cells did increase with induced senescence (panel R, compare bars 3 and 4), the increase was significantly decreased compared with migration of HMECs post-co-culture with RMF cells (panel R, compare bars 2 and 4). This is consistent with the results in FIG. 16 panel A showing that OPN increases to a much smaller degree in +Tiam1-RMF cells undergoing senescence than in control RMF cells with endogenous Tiam1 levels.

### Example 29

### Down-Regulation of OPN in Senescent RMF Cells Inhibits the Invasion and Migration of Associated Epithelial Cells

We also asked whether blocking the up-regulation of OPN in senescent cells would inhibit the increased epithelial cell invasiveness induced by senescent fibroblasts by performing similar assays using an RMF line with stable silencing of OPN (shOPN-RMF). In co-cultures of HMECs with senescent shOPN-RMF there was blunting in numbers of projections per spheroid compared with senescent control RMFs, with increased numbers of spheroids with 0-1 projection and decreased number of spheroids with projections (FIG. 18, panels A-P, compare panels G and O; panel Q, compare bars 2 and 4). Migration of HMECs isolated from co-cultures with shOPN-RMF did increase with induced senescence (FIG. 18, panel R, compare bars 3 and 4), but this increase was also significantly decreased compared with migration of HMECs post-co-culture with control RMFs (panel R, compare bars 2 and 4). These results show that inhibition of OPN, like up-regulation of Tiam1, partially blocks the increased invasiveness induced by senescent fibroblasts.

### Example 30

### OPN Mediates the Effects of Tiam1-Deficiency in Fibroblasts on Associated Epithelial Cells

As OPN is a secreted glycoprotein, results using a modified transwell migration assay were sought. Senescent fibroblasts pre-seeded into the bottom chamber were observed to induce increased migration of HMECs across a membrane, compared with pre-senescent fibroblasts (FIG. 19 panel A, compare bars 1 and 2). Fibroblasts with OPN silencing induced less migration at baseline (compare bars 1 and 3), and almost no increase in migration when rendered senescent (compare bars 3 and 4). This is consistent with the results seen using co-cultures, with decreased epithelial cell invasion into matrix and migration post-co-culture with OPN-deficient fibroblasts (FIG. 18).

This assay was used to test the effect of inhibiting OPN secretion in Tiam1-deficient fibroblasts. Similar to senescent fibroblasts, Tiam1-deficient fibroblasts pre-seeded in the bottom chamber induced increased migration of HMECs across a membrane compared with fibroblasts with intact Tiam1 levels (FIG. 19 panel B, compare bars 1 and 3). Incorporation of an anti-osteopontin antibody into the bottom chamber blocked the increased migration induced by Tiam1-deficient fibroblasts (FIG. 19 panel B, compare bars 3 and 4). In addition, concurrent silencing of OPN in Tiam1-deficient fibroblasts also blocked the increased migration induced by Tiam1-deficient fibroblasts (FIG. 19 panel C, compare bars 2 and 3). These data show that Tiam1 deficiency in fibroblasts promotes epithelial migration and invasion through up-regulation of osteopontin.

Taken together with our work on the effects of Tiam1 silencing in tumor-associated fibroblasts, these findings indicate that one mechanism by which senescent fibroblasts promote neoplastic progression in associated tumors is through degradation of fibroblast Tiam1 protein and consequent increase in fibroblast secretion of osteopontin. As herein, Tiam1-deficient fibroblasts promote invasion and metastasis of associated epithelial tumor cells using both in vitro and in vivo models. Further, examples using the in vitro three-dimensional culture model of cellular invasiveness have elucidated several steps underlying this effect. Thus, stress-induced senescence leads to decreased Tiam1 protein and increased expression of osteopontin, that lysates from senescent cells induce Tiam1 protein degradation in a calcium and calpain-dependent fashion. Further, Tiam1 protein levels lead to converse changes in osteopontin mRNA and protein secretion. Increasing the Tiam1 expression level in cells blunts the rise in osteopontin upon senescence induction. Senescent fibroblasts induce increased invasion and migration in co-cultured mammary epithelial cells. These effects in the epithelial cells are ameliorated by either increasing the Tiam1 or decreasing the osteopontin in the fibroblasts. In a seeded cell migration assay either senescent fibroblasts or Tiam1-deficient fibroblasts induce increased epithelial cell migration that was dependent on fibroblast secretion of osteopontin.

Post-transcriptional regulation of Tiam1 includes protein cleavage and degradation. Tiam1 has tandem N-terminal PEST sequences, defining it as a potential target for rapid proteolytic cleavage (Belizario, J E, et al., 2008, Curr Protein Pept Sci 9:210-20; Rechsteiner, M, et al., 1996, Trends Biochem Sci 21:267-716). Tiam1 undergoes caspase-mediated cleavage in cell lines undergoing apoptosis [Qi H, et al., 2001, Cell Growth Differ 12:603-11]. Calpain-mediated degradation was recently shown to be the likely mediator of Src-induced Tiam1 depletion localized to adhere junctions in MDCK cells [Woodcock S A, et al., 2009, Mol Cell 33:639-53]. Calpains are a family of 14 calcium-regulated cysteine proteases and 2 regulatory proteins that initiate precise limited substrate proteolysis [Franco S J, et al. 2005, J Cell Sci 118:3829-38]. Over 100 diverse calpain targets have been identified to date, indicating a wide role for calpains in mediating signal transduction processes. Calpain proteases are likely involved in the DNA damage response initiated at the start of cellular senescence, as depletion of the CAPSN1 regulatory subunit diminished senescence markers including phosphorylated histone H2AX in cells induced to undergo senescence through oncogenic, radiation, or chemical stress [Demarchi F, et al., 2010, Cell Cycle 9:755-60]. Examples herein show calpain activity is increased in cells undergoing senescence and that Tiam1 is likely to be a calpain target in cells under those conditions.

Cellular senescence is thought to serve as a tumor-suppressor response in proliferating tissues that limits the replication of cells with DNA damage or telomere dysfunction and is also thought to contribute to aging (reviewed in [Campisi J, et al., 2007, Nat Rev Mol Cell Biol 8:729-40]). The number of senescent cells increases with age and age-dependent p16-mediated suppression of progenitor cell proliferation has been demonstrated in mouse brain, bone marrow, and hematopoietic tissues [Janzen V. et al., 2006, Nature 443:421-6; Krishnamurthy J, et al., 2006, Nature 443:453-7; Molofsky A V, et al., 2006, Nature 443:448-52; Zindy F, et al., 1997, Oncogene 15:203-11]. In contrast to the tumor-suppressor function of senescence, in various models of the tumor microenvironment senescent fibroblasts confer a paradoxic increase in neoplastic progression in associated tumors, with multiple cytokines, growth factors, and matrix-altering enzymes implicated as potential mediators [Bavik C, et al, 2006, Cancer Res 66:794-802; Coppe J P, et al., 2006, Journal of Biological Chemistry 281:29568-74; Dilley T K, et al., 2003, Exp Cell Res 290:38-48; Parrinello S, et al., 2005, J Cell Sci 118, 485-96]. The altered pattern of gene expression exhibited by senescent cells is associated with increased secretion of inflammatory cytokines that alter the tissue microenvironment through disruption of normal architecture and stimulation of neighboring cells (Rodier et al., 2009). The model herein utilizes immortalized fibroblasts, and also cells undergoing stress-induced senescence (SIPS) rather than replicative senescence (RS). SIPS cells and RS cells share a number of features, including morphology, SA-βgal staining, inability to replicate in response to various growth factors, similar changes in p53/p21 and p16^{INK-4a} pathways, and significant similarities in gene expression patterns [Chen Q, et al., 1995, Proc Natl Acad Sci USA 92:4337-41; Toussaint O, et al., 2000, Exp Gerontol 35:927-45]. In addition, both SIPS cells and RS fibroblasts demonstrate increased OPN and can stimulate the growth of preneoplastic cells [Bavik C, et al, 2006, Cancer Res 66:794-802; Krtolica A, et al., 2001, Proc Natl Acad Sci USA 98:12072-7; Pazolli E et al., 2009, Cancer Res 69:1230-9]. Finally, the accumulation of senescent cells with aging may result from tissue damage due to oxidative stress from reactive oxygen species, suggesting considerable overlap between experimentally induced SIPS cells (especially with oxidative stress) and RS cells [Krtolica A, et al., 2002, Int J Biochem Cell Biol 34:1401-14; Toussaint O, et al., 2000, Exp Gerontol 35:927-45]. Results in examples herein may thus be relevant to cells undergoing senescence as a result of aging or exposure to stressors such as chemotherapy, radiation, or chronic inflammation.

Increased fibroblast secretion of osteopontin shown herein is an important mechanism underlying the effect of senescent and/or Tiam1-deficient fibroblasts in promoting increased invasion and migration of associated mammary epithelial cells. OPN induces multiple effects in multiple cell types. In breast cancer cells OPN is reported to regulate inhibition of apoptosis through up-regulation of NFκB and PI3K pathways, increased invasiveness through up-regulation of NFκB, matrix metalloproteinase-2, and urokinase plasminogen activator, and increased migration dependent on EGF and HGF receptors (reviewed in [Wai P Y, et al., 2004, J Surg Res 121:228-41]). Many OPN effects are triggered through ligation of αᵥβ integrin and CD44 receptor families. Tiam1 itself is involved in multiple signaling pathways through interactions with scaffold proteins that direct Tiam1-mediated Rac activation to specific downstream pathways [Rajagopal S, et al., 2010, J Biological Chemistry 285:18060-71]. It is likely that only a subset of Tiam1 pathways contribute to OPN regulation, as silencing the Rae GTPase itself does not completely phenocopy Tiam1 deficiency in fibroblasts [Xu K, et al., 2010, Oncogene 29:6533-42]. The method described here for co-culture with specific cell populations and isolating post-co-culture epithelial cells will allow for systematic analysis of the effects of micro-environment fibroblasts on specific Tiam1 pathways, specific OPN receptors, and potential target pathways in turn.

Without being limited by any particular theory or mechanism of action, a pathway is postulated herein by which senescent fibroblasts in the tumor microenvironment facilitate invasiveness of associated mammary epithelial cells through degradation of fibroblast Tiam1, which leads to increased fibroblast secretion of osteopontin. A technique is provided for isolating epithelial cells exposed to microenvironment fibroblasts, specific steps involved in how Tiam1 protein level regulates osteopontin, and how fibroblast osteopontin modulates mammary epithelial cell invasiveness. This method has the potential to be used to identify possible targets for therapeutic inhibition of microenvironment-induced tumor invasiveness.

### Example 31

### Validation of Tiam1 Predictive Value Using Retrospective Samples

A monoclonal anti-Tiam1 antibody for immunohistochemistry is predicted to be useful as a prognostic biomarker for women with high-grade DCIS. A retrospective study of banked tumor specimens of women having a diagnosis of high-grade DCIS at a time point at least 10 years ago, and for whom long-term follow-up outcomes (recurrent breast cancer or no recurrence) are known is performed. Depending on numbers of available specimens, the analysis is either a retrospective case control study or a retrospective cohort study. Under the assumption that recurrence rates are 5% if Tiam1 is expressed in tumor-associated fibroblasts and 30% if Tiam1 is not expressed in tumor-associated fibroblasts, a case control study having 19 cases with recurrence and 19 cases without recurrence yields 80% power. Under similar assumptions, a cohort study requires examination of 101 cases for 80% power. All cases of high-grade DCIS are included, with results stratified for the ratio of estrogen receptor/progesterone receptor (ER/PR) and Her2 expression status since these are known clinically relevant breast cancer markers. These data allow derivation of positive and negative predictive values for the assay.

### Example 32

### Use of Antibody for Prognosis to Select Patients for Prospective Aggressive Therapy

Tiam1 expression in tumor-associated fibroblasts is shown in the Examples above to be sufficiently prognostic using the monoclonal antibody, therefore this method establishes whether women with poor-prognosis DCIS by this assay would benefit from more aggressive adjuvant therapy at the time of diagnosis. This more aggressive adjuvant therapy uses standard chemotherapy, targeted therapy such as trastuzumab in the case of Her2-positive DCIS, anti-angiogenic therapy such as with bevacizumab, or other targeted therapy under development for the treatment of breast.

A prospective randomized trial is used to stratify subjects with high-grade DCIS according to Tiam1 fibroblast expression. Women with high Tiam1 expression are administered treatment according to standard of care (SOC: surgery+/−radiation, with adjuvant estrogen blockade for ER+DCIS). Women with low Tiam1 expression are randomized to SOC or SOC with more intensive adjuvant therapy with one of the above therapeutic approaches.

As shown herein, a modified human breast cancer mouse model demonstrates the pro-invasive/metastatic effect of Tiam1-deficient mammary fibroblasts. This model is used in pre-clinical trials to determine which specific therapeutic approach best overcomes the pro-invasive effects of Tiam1-deficient fibroblasts. The results of these trials are applied to the design of the prospective anti-cancer agent protocols. Determining which cases of high-grade DCIS are most likely to recur due to or associated with Tiam1-deficiency in fibroblasts, and identifying which therapeutic agent(s) best overcome the effects of Tiam1 deficiency, optimizes the likelihood that such an intervention successfully decreases the breast cancer recurrence rate. This strategy distinguishes this approach from less-targeted approaches used in the past that have not been fruitful to date in determining optimal treatment in high-grade DCIS.

Tiam1 is expressed in many tissues. It is likely that Tiam1 in tumor-associated fibroblasts plays a role in regulating the invasiveness and metastatic potential of many other cancer types. Thus the anti-Tiam 1 antibody is evaluated as a useful prognostic biomarker in other malignancies where a diagnosis of intra-epithelial dysplasia/neoplasia is associated with uncertain clinical significance (such as cervical, prostate, and oro-pharyngeal malignancies), or where use of a prognostic biomarker could aid therapeutic decision-making in early-stage cancers, such as colorectal cancer.

While the technology has been particularly shown and described with reference to specific embodiments, it should be understood by those skilled in the art that various changes in form and detail may be made therein without departing from the spirit and scope of the technology as defined by the appended claims.

1. A method for evaluating potential for invasiveness, metastasis, or recurrence of an epithelial cell cancer, the method comprising:
detecting Tiam1 expression in a tissue sample, wherein the tissue sample includes fibroblasts and tumor cells or suspected tumor cells, and the detecting includes at least one of amount and location of the Tiam1; and,
assessing Tiam1 expression level in fibroblasts adjacent to tumor cells, wherein a decreased level of Tiam1 expression in the fibroblasts adjacent to tumor cells, in comparison to a control non-invasive standard or to a sample taken at a different point in time, is indicative of increased potential of at least one of the invasiveness, metastasis, or recurrence of the epithelial cell cancer.

2. The method according to claim 1 further comprising prior to detecting, obtaining the tissue sample from a subject having or suspected of having at least one selected from the group of: an epithelial cell cancer, risk for developing an epithelial cell cancer, a risk for developing the cancer arising from family history or genetic analysis, a remission from the cancer, and a risk for developing a recurrence of the cancer.

3. The method according to claim 2, wherein the epithelial cell cancer is at least one selected from breast; prostate; lung; bladder; uterine; ovarian; brain; head and neck; esophageal; pancreatic; gastric; germ cell; and colorectal cancers.

4-5. (canceled)

6. The method according to claim 1 wherein detecting Tiam1 expression is detecting Tiam1 protein or detecting Tiam1 RNA by at least one selected from the group of: contacting the sample with an anti-Tiam1 antibody, using immunohistochemistry, hybridizing in situ a tissue or a cell using a nucleic acid probe, performing quantitative real-time polymerase chain reaction, immunoblotting an electrophotogram, and using a detection reagent.

7. (canceled)

8. A method for modulating invasiveness and metastatic potential of an epithelial cell cancer cell comprising contacting fibroblasts associated with the epithelial cell cancer with a reagent that causes increased expression of Tiam1.

9. The method according to claim 8, wherein the reagent is selected from: a low molecular weight drug; a vector carrying a gene or a portion thereof encoding a Tiam1 protein; and a naked nucleic acid encoding the protein.

10. A method for screening compounds to identify an agent that modulates potential for invasiveness, metastasis, or recurrence of an epithelial cell cancer comprising:
contacting fibroblasts with at least one candidate compound, and
assessing at least one of Tiam1 expression levels or osteopontin (OPN) expression levels in resulting contacted fibroblasts, wherein altered expression of Tiam1 or OPN in the contacted fibroblasts in comparison to fibroblasts not so contacted and otherwise identical, identifies the agent that modulates the potential for the invasiveness, metastasis, or recurrence of the epithelial cell cancer.

11. The method according to claim 10, wherein the fibroblasts are associated with the epithelial cell cancer.

12. The method according to claim 10, wherein the fibroblasts are cultured in vitro.

13. The method according to claim 10 wherein the fibroblasts are associated with tumor cells in a three-dimensional spheroid system.

14. The method according to claim 10 wherein the fibroblasts are associated with tumor cells in situ in an animal, and the method further comprises a pre-clinical evaluation of the agent.

15. The method according to claim 12, wherein the method further comprises, prior to contacting, stressing the fibroblasts wherein the fibroblasts develop senescence.

16. The method according to claim 15, wherein stressing comprises exposing the fibroblasts to an agent selected from: an oxidizing agent; a mutagen; a carcinogen; and radiation.

17. The method according to claim 15, further comprising measuring an extent of reversing or preventing senescence.

18. A method of using a three-dimensional spheroid cell culture comprising epithelial cells and fibroblasts in an extracellular matrix, for prognosis of an epithelial cancer, comprising culturing the epithelial cells and the fibroblasts in the matrix, wherein the epithelial cells and the fibroblasts aggregate to form the three-dimensional spheroids, and recovering the spheroids from the matrix and analyzing the epithelial cells for invasiveness or the fibroblasts for altered gene expression or protein expression.

19. The method according to claim 18, wherein the fibroblasts are human.

20. The method according to claim 18, wherein the fibroblasts are obtained from a cancer patient tumor biopsy or from a normal subject reduction mammoplasty sample.

21. The method according to claim 18, further comprising prior to culturing, recombinantly engineering at least one of the epithelial cells and the fibroblasts.

22. The method according to claim 18, further comprising recombinantly modulating expression of at least one gene in at least one of the epithelial cells and the fibroblasts.

23. The method according to claim 18, wherein modulating expression of at least one gene further comprises modulating expression of Tiam1 or osteopontin in the fibroblasts, and wherein the method further comprises analyzing invasiveness of the epithelial cells into the extracellular matrix in comparison to control fibroblasts in which gene expression is not modulated and the fibroblasts are otherwise identical.

24. The method according to claim 18, wherein the matrix is at least one selected from: BD Matrigel, AlphaMAX 3D, alphaGEL3D, Porocell, BD PuraMatrix, AlgiMatrix, PathClear, Geltrex, MaxGel, HydroMatrix, Mebiol Gel3D, Alvetex, MAPTrix and Cultrex.

25. (canceled)

26. A kit for evaluating potential for invasiveness, metastasis, or recurrence of an epithelial cell cancer, the kit comprising:
a detection reagent suitable for detecting presence of protein T-cell lymphoma invasion and metastasis-inducing protein 1 (Tiam1) or an amount of a gene product of a gene encoding the TIAM1 in cells of a tissue sample, wherein the tissue sample includes tumor tissue of the cancer and cell tissue surrounding the tumor, and
instructions for using the detection reagent to detect Tiam1 or the amount of the gene product in tumor cells of the tissue sample, and in fibroblasts of the tissue sample associated with the tumor, thereby evaluating the potential for the invasiveness, metastasis, or recurrence of the cancer based on amounts of Tiam1 or the amount of the gene product in the tumor cells and in the fibroblasts.

27. The kit according to claim 26, wherein the instructions include statistical correlations for evaluating the expression of a low amount of Tiam1 in the fibroblasts surrounding the tumor as an indication of a greater likelihood that the tumor is invasive or has greater potential for the invasiveness, metastasis, or recurrence, and a high amount of Tiam1 in the fibroblasts surrounding the tumor as an indication that the tumor is non-invasive, or has less potential for the invasiveness, metastasis, or recurrence.

28. The kit according to claim 26, the detection reagent is an antibody that specifically binds to Tiam1.

29-54. (canceled)
